(19) United States
(12) Patent Application Publication
(10) Pub. No.: US 2024/0065554 A1
(43) Pub. Date: Feb. 29, 2024

(54) SYSTEMS AND METHODS FOR SKIN ANALYSIS

(71) Applicant: BARACODA DAILY HEALTHTECH, Neuilly-sur-Seine (FR)

(72) Inventors: Thomas SERVAL, Paris (FR); Ali MOUIZINA, Paris (FR)

(21) Appl. No.: 18/260,563

(22) PCT Filed: Jan. 11, 2022
(86) PCT No.: PCT/IB2022/050159

§ 371 Date: Jul. 6, 2023

Related U.S. Application Data
(60) Provisional application No. 63/135,911, filed on Jan. 11, 2021.

(51) Int. Cl. A61B 5/00; A47G 1/02; A61B 5/01; G06T 5/00; G06T 5/20; G06T 7/00; G06V 10/764; G06V 10/77; G06V 20/20; G06V 20/64; G06V 40/10

(52) U.S. Cl. CPC A61B 5/0079; A47G 1/02; A61B 5/01; A61B 5/444; A61B 5/7253; A61B 5/7425; G06T 5/001; G06T 5/20; G06T 7/0014; G06V 10/764; G06V 10/7715; G06V 20/20; G06V 20/64; G06V 40/10; A61B 2090/365; A61B 2562/0233; A61B 2562/0257; A61B 2562/0271

(57) ABSTRACT

Systems and methods are provided for performing skin analysis using smart mirror systems. In one example, a plurality of RGB images are acquired via an RGB camera integrated with a smart mirror. Using the RGB images as input, multiple skin key performance indicators (KPIs, also referred to as skin features) including sebum porphyrin, redness, acne, eczema, rosacea, UV spots, brown spots, eye bags, scars are classified and/or quantified. In particular, computer vision algorithms are used in order to detect tone changes within the user's skin, and segment the various skin features. Thus, the systems and methods describes herein are robust in adapting to different skin colors, tones, and/or hues.

### CROSS REFERENCE TO RELATED APPLICATION

This application claims the benefit of and priority to U.S. Provisional Application No. 63/135,911, filed Jan. 11, 2021, which is hereby incorporated by reference herein in its entirety.

### TECHNICAL FIELD

The present disclosure relates to an interactive mirror device, more specifically, the present disclosure relates to an interactive mirror with the ability to perform skin analysis.

### BACKGROUND

A smart mirror may assist a user in their daily routine. For example, the smart mirror may assist the user by automatically adjusting lighting intensity for better visualization, provide tutorials for various make-up and hair styling routines, as well as provide make-up and styling suggestions to the user. An example smart mirror system typically includes a camera and a display integrated with a mirror. However, existing smart mirror systems are cannot be used for cosmetic and/or medical condition monitoring or analysis. For example, a user is unable to evaluate if a treatment for a skin condition (e.g., wrinkle) has been effective by looking at the smart mirror, even under adjusted lighting conditions. Further, some skin changes and/or physiological changes that are subtle and/or slow cannot be easily visualized by looking at a camera image.

### SUMMARY

Systems and methods are provided for performing skin analysis. In particular, systems and methods are provided for performing skin analysis using a smart mirror device. As mentioned above, existing smart mirror systems are ineffective for analyzing and/or monitoring various skin conditions. The inventors herein have identified the above-mentioned disadvantages of existing smart mirror systems. Accordingly, in one example, some of the above issues may be at least partially addressed by a smart mirror system comprising a frame; a mirror coupled to the frame; a camera coupled to the frame; and a first light source configured to output white light having wavelengths in a visible range; a second light source configured to output UV light having wavelengths in a UV range; one or more processors including executable instructions stored in one or more non-transitory memory devices that when executed cause the one or more processors to: acquire one or more images of a user via the camera using a first lighting condition provided by the first light source or the second light source; process the one or more images to detect one or more skin features; generate a skin profile analysis output based on the one or more skin features; and display, via a display portion of a user interface of the mirror, the skin profile analysis output.

In this way, a skin profile is generated that includes information regarding skin response to a range of wavelengths in the electromagnetic spectrum. The skin profile may then be used for evaluation and monitoring of variety of skin conditions, including medical and/or cosmetic conditions, and/or treatments.

According to implementations of the present disclosure, a smart mirror system comprises a frame; a mirror coupled to the frame; a camera coupled to the frame; a first light source configured to output white light having wavelengths in a visible range; a second light source configured to output UV light having wavelengths in a UV range; and one or more processors including executable instructions stored in one or more non-transitory memory devices that when executed cause the one or more processors to: acquire a first set of images of a user via the camera using a first lighting condition provided by the first light source; acquire a second set of images of the user via the camera using a second lighting condition provided by the second light source; process the first set of images to obtain a first set of skin features; process the second set of images to obtain a second set of skin features; generate a skin profile analysis output based on one or more of the first set of skin features and the second set of skin features.

In another implementation, a method for performing skin analysis, the method comprises acquiring, via a camera integrated with a smart mirror, one or more images of a user illuminated under a first lighting condition provided by a first light source coupled to the smart mirror; processing, via a processor of the smart mirror, the one or more images to classify one or more skin features; generating, via the processor, a skin profile analysis output based on the one or more skin features; and outputting, via a display portion of a user interface of the mirror, the skin profile analysis output.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The foregoing and other advantages of the present disclosure will become apparent upon reading the following detailed description and upon reference to the drawings.

FIG. 1A is a block diagram of a smart mirror system, according to an embodiment of the present disclosure;

FIG. 1B is a block diagram of an image processing system for performing skin analysis, according to an embodiment of the present disclosure;

FIG. 2 is a flowchart illustrating an example method for performing skin analysis using the smart mirror system of FIG. 1A, according to an embodiment of the present disclosure;

FIG. 3 is a flowchart illustrating an example method for evaluating a skin condition using the smart mirror system of FIG. 1A, according to an embodiment of the present disclosure;

FIG. 4 is a flowchart illustrating another example method for performing skin analysis using the smart mirror system of FIG. 1A, according to an embodiment of the present disclosure;

FIG. 5A is a flowchart illustrating an example method for performing skin analysis using the smart mirror system of FIG. 1A, according to an embodiment of the present disclosure;

FIG. 5B is a flowchart illustrating and example method for quantification during skin analysis, according to an embodiment of the present disclosure;

FIG. 6 shows a table illustrating one or more parameters for performing skin condition analysis using the smart mirror system of FIG. 1A, according to an embodiment of the present disclosure;

FIG. 7A shows an example image depicting visualization of skin extraction for facial skin analysis; according to an embodiment of the present disclosure;

FIG. 7B shows an enlarged portion of FIG. 7A depicting visualization of a plurality of skin clusters after transforming to space and L*a*b (LAB) color domain;

FIG. 7C shows an example histogram after dimension reduction using principal component analysis for the image portion shown at FIG. 7B;

FIG. 7D shows an example visualization of redness detection on the image portion shown at FIG. 7B;

FIG. 8A shows another portion of FIG. 7A depicting visualization of a plurality of skin cluster after transforming to space and LAB color domain;

FIG. 8A;

FIG. 8B shows an example dominant color identified in the image portion of FIG. 8C shows superposition of masks on the redness areas in the image portion of FIG. 8A;

FIG. 8D shows an example output after applying a filter on the image portion of FIG. 8C; and

FIG. 8E shows an example output after determining the final distance to skin tone on the image portion of FIG. 8D in the dominant redness areas with mask;

FIG. 9 is a perspective view of an example smart mirror system, according to an embodiment of the present disclosure;

FIG. 10A is a front elevation view of the smart mirror system of FIG. 9, according to an embodiment of the present disclosure; and

FIG. 10B is a side elevation view of the smart mirror system of FIG. 9, according to some implementation of the present disclosure.

While the present disclosure is susceptible to various modifications and alternative forms, specific implementations and embodiments have been shown by way of example in the drawings and will be described in detail herein. It should be understood, however, that the present disclosure is not intended to be limited to the particular forms disclosed. Rather, the present disclosure is to cover all modifications, equivalents, and alternatives falling within the spirit and scope of the present disclosure as defined by the appended claims.

### DETAILED DESCRIPTION

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

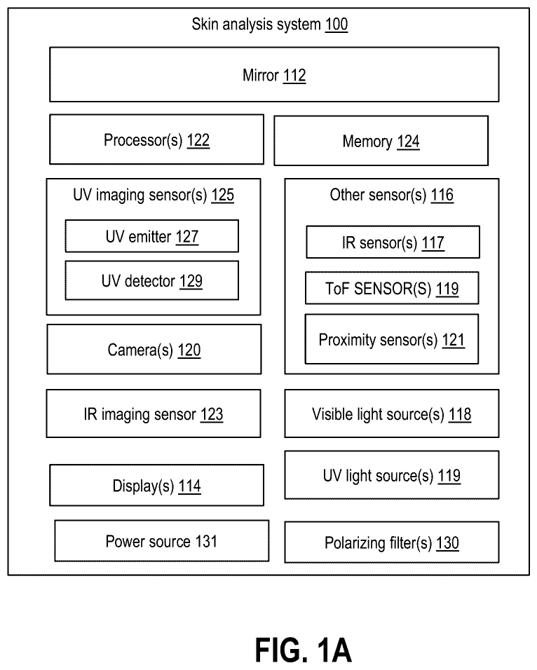

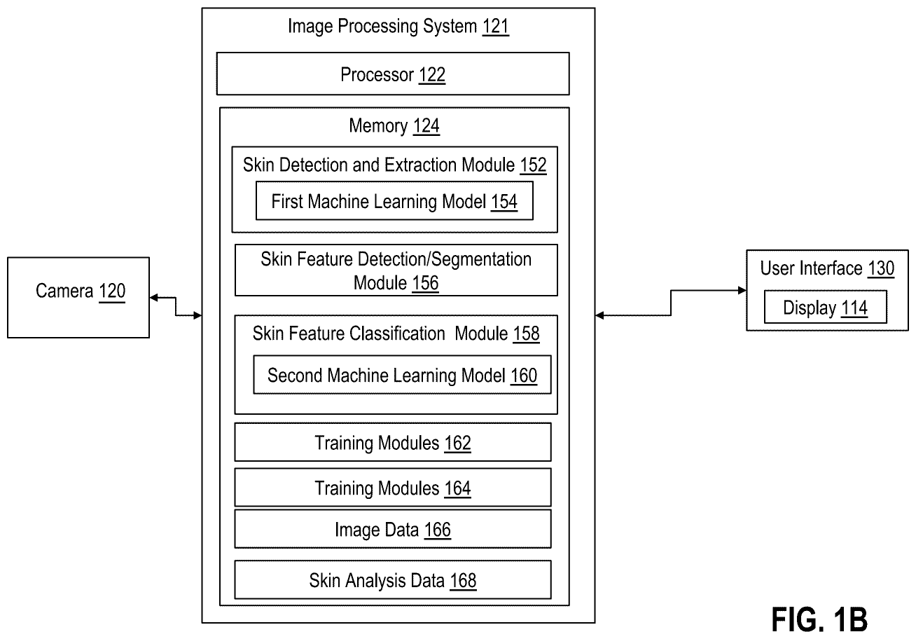

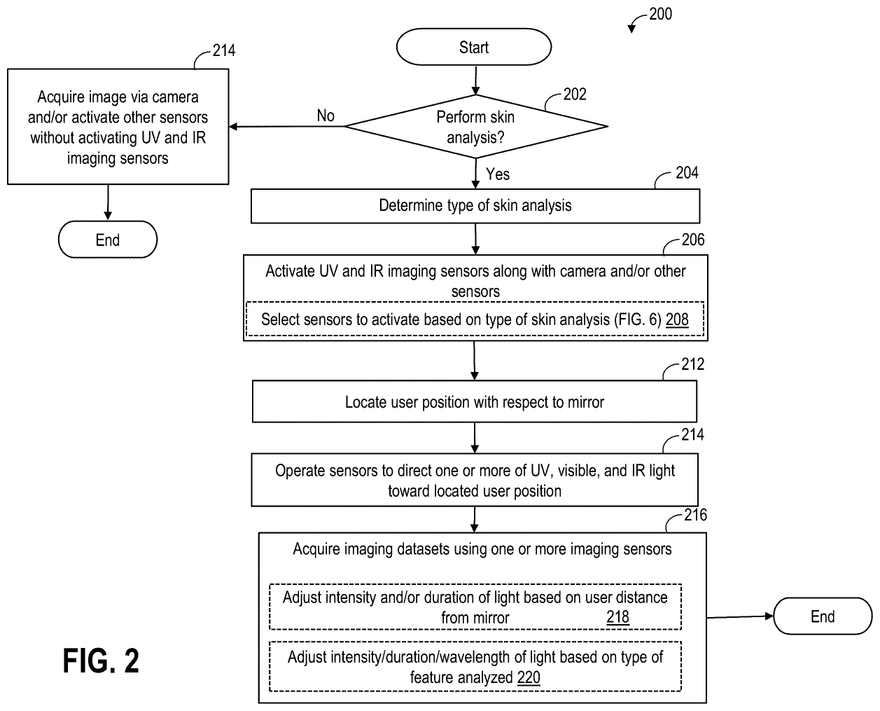

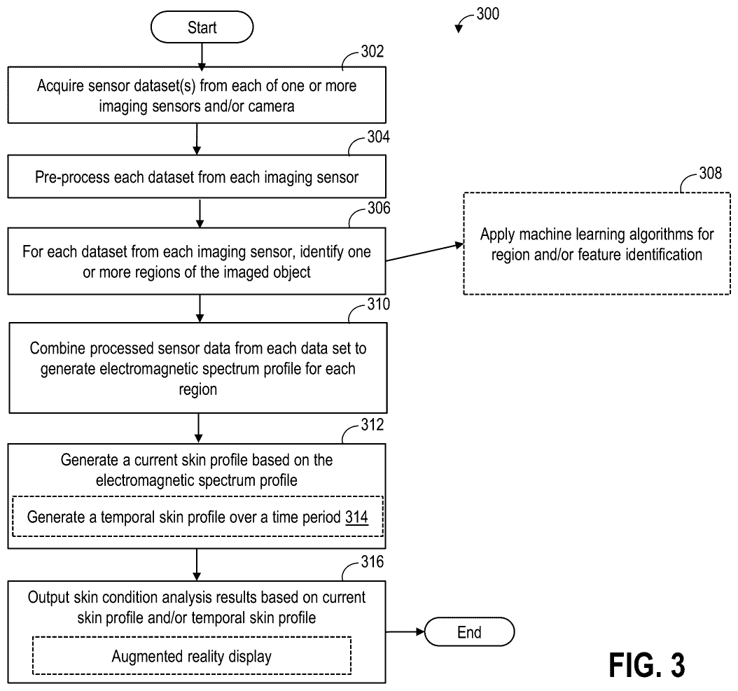

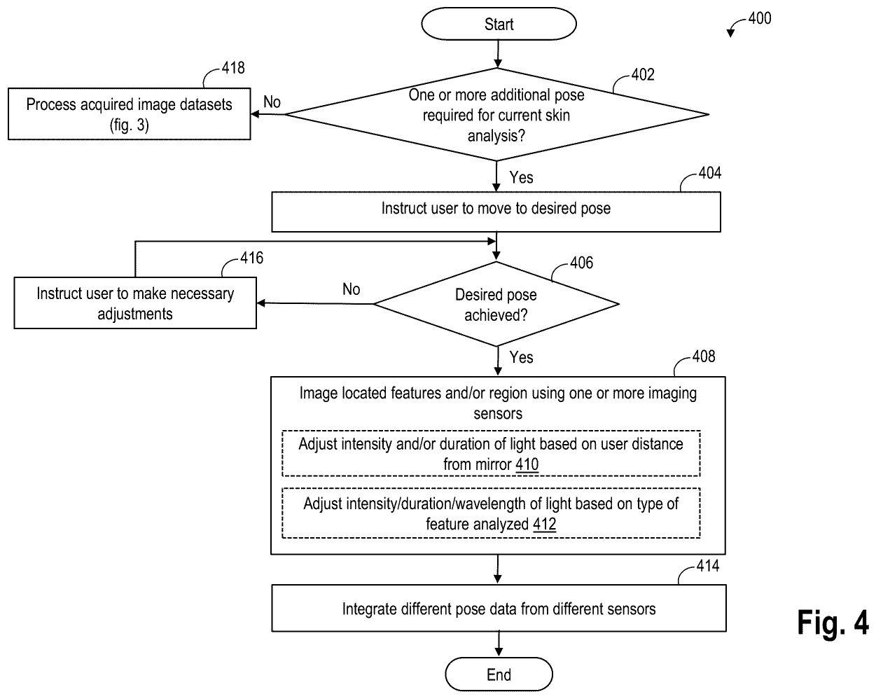

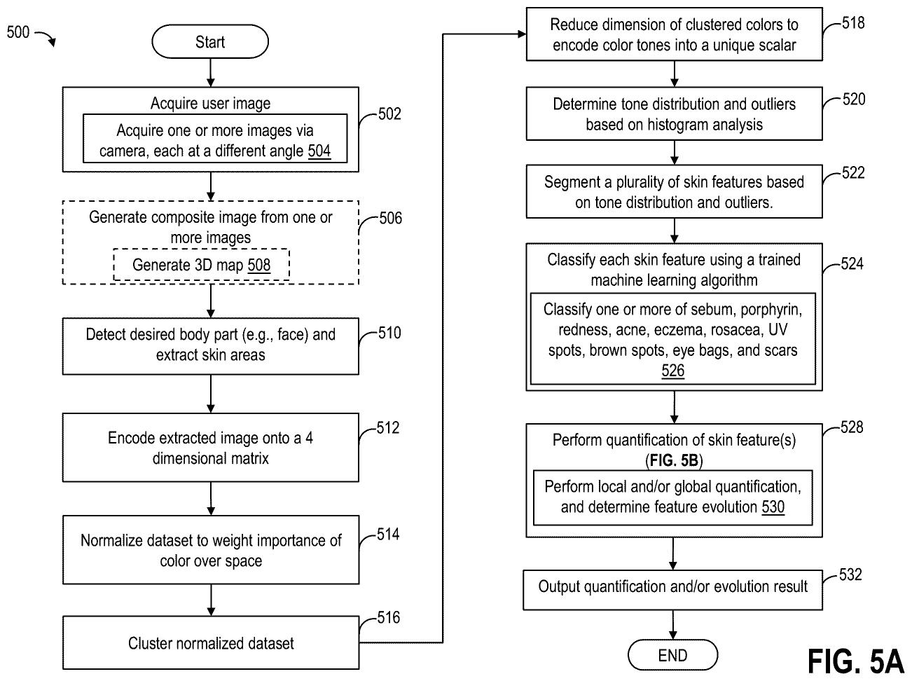

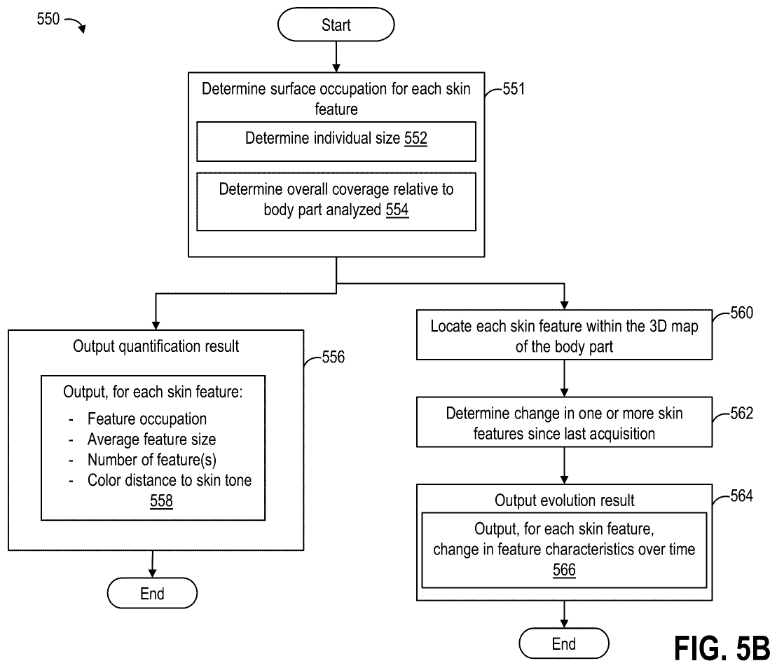

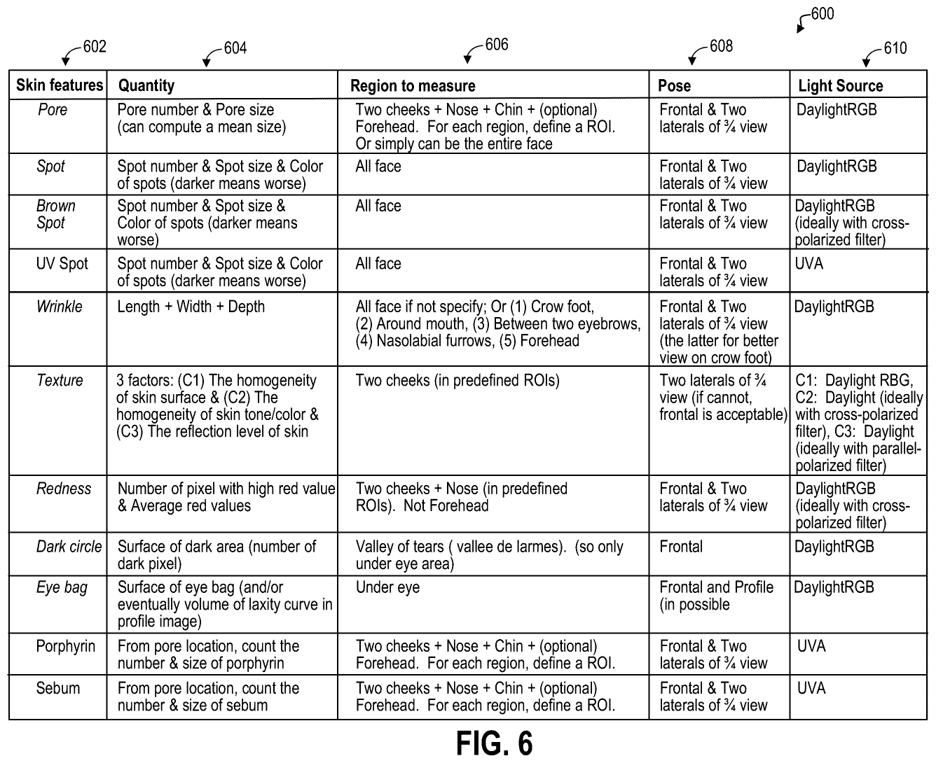

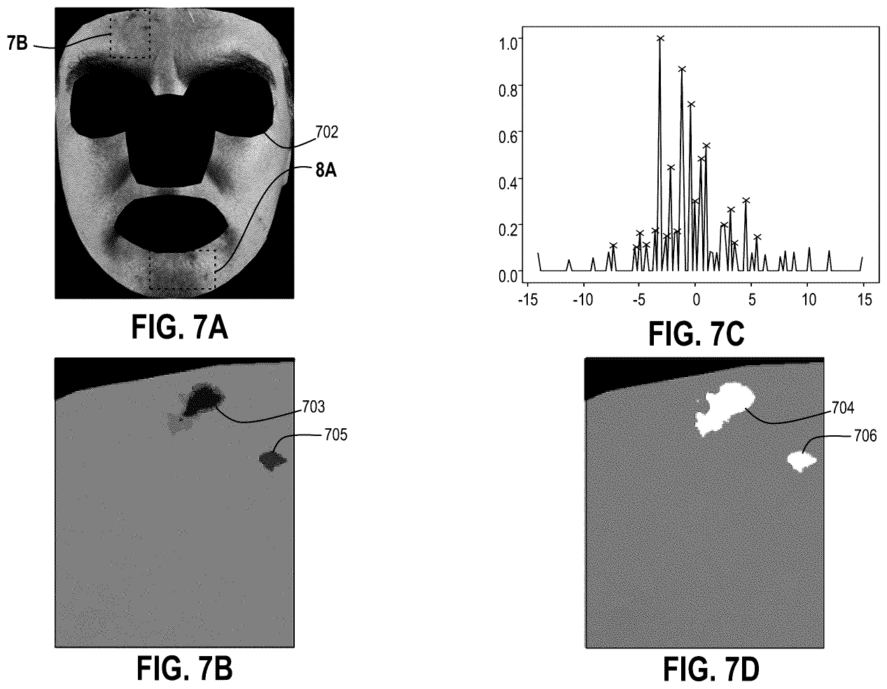

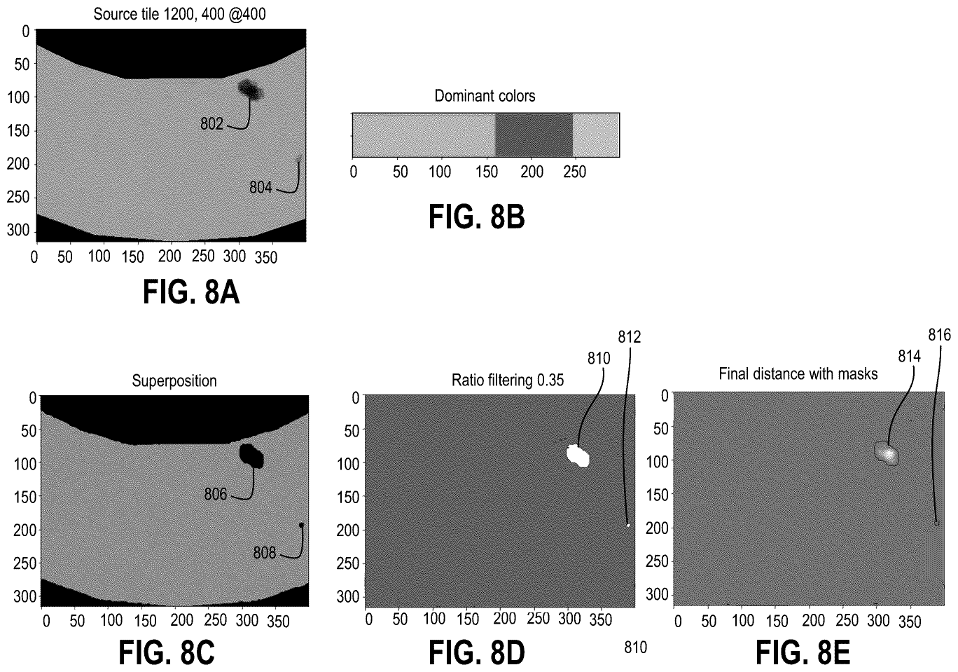

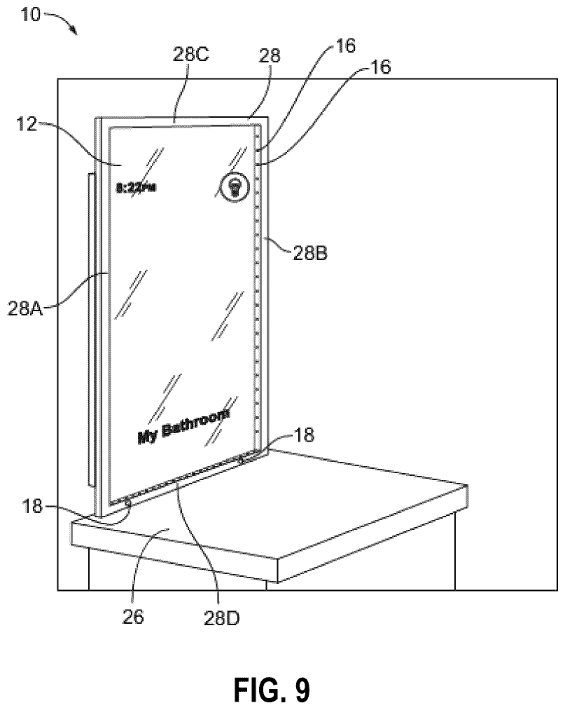

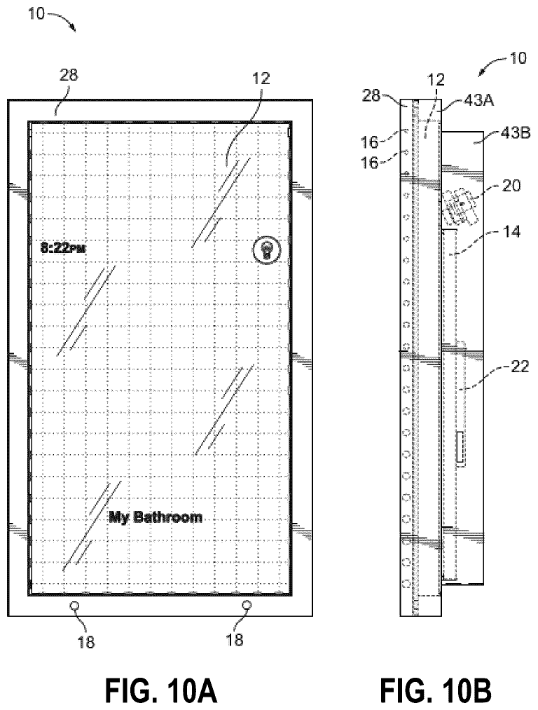

The systems and methods are provided for performing skin analysis to detect and quantify one or more skin features from one or more images of a user acquired via a camera. In particular, systems and methods are provided for performing skin analysis on a variety of skin colors, tones, and/or hues. In one embodiment, skin analysis may be performed using a smart mirror system. An example block diagram of a smart mirror system is shown at FIG. 1A and an example image processing system that may be implemented with the smart mirror system is shown at FIG. 1B. Further, an example schematic of a smart mirror system is described at FIGS. 9, 10A, and 10B. Example methods of initiating skin analysis and/or acquiring images (or imaging datasets) are shown at FIGS. 2 and 4. Further, an example method for generating a skin profile for a user using skin features derived from images taken under different lighting conditions (e.g., white light, UVA, etc.) is shown at FIG. 3. In particular, a skin analysis map of a body part (e.g., a 2D or a 3D face map) is generated, wherein each pixel group or voxel group in the map includes a reference to corresponding response features identified using UV light and/or visible light. Thus, skin features identified using visible light are mapped onto the 2D or 3D face map, and the skin features identified using UV light are also mapped onto the 2D or 3D face map. Thus, each region in the 2D or 3D face map includes a reference to UV light features as well as visible light features. During subsequent analysis over time, the 2D or 3D face map may be used to track evolution of any given skin feature over time. Further, an example method for identifying skin areas, detecting and classifying various skin features, and global, local, and evolutional quantification of skin is described at FIGS. 5A, 5B, and 6. Examples visualizations at various steps in the skin analysis method is shown in one example at FIGS. 7A-7D, and in another example at FIGS. 8A-8E.

Technical advantages of the methods and systems described herein include improved accuracy and efficiency in the determination of local and global aspects of one or more skin features, such as a size of each skin feature (redness, sebum, wrinkles etc.), a distribution of each skin feature, and an overall skin analysis profile. Further, systems and methods provide improvement in efficiency and accuracy in detecting feature changes through time. Further, the methods and systems described herein allows a skin feature to be evaluated with respect to surrounding skin areas (that is, normal skin areas). That is, the methods and systems described herein provide relative skin color and tonal assessment. Thus, the methods and systems described herein may be adapted to various human skin tones, hues, and/or color. Thus the systems and methods described herein provide increased adaptability to a wide range of users. Further still, the systems and methods described herein provide a comprehensive analysis of skin features. Taken together, the systems and methods described herein provide significant improvement in the area of skin analysis and smart mirror systems.

Referring to FIG. 1A, a skin analysis system 100 includes a mirror 112, at least one processor 122, and at least one memory 124. The skin analysis system 100 further includes a plurality of sensors.

The plurality of sensors may include one or more ultraviolet (UV) imaging sensors 125. In one example, the one or more UV imaging sensors 125 may be active UV sensors that include one or more UV emitters 127 (e.g., UV LED) for emitting UV light and one or more UV detectors 129 for detecting UV reflected light. In another example, the one or more UV imaging sensors 125 may be passive sensors that include one or more UV detectors 129. In such examples, an external source of UV may be used.

The plurality of sensors may include one or more infrared (IR) imaging sensors 123, which may be active or passive sensors. When configured as active sensors, the IR imaging sensors 123 may include one or more IR emitters and one or more IR receivers. When configured as passive sensors, the IR sensors may measure IR light radiating from one or more objects in a field of view of the IR sensor.

In some implementations, the plurality of sensors may include one or more red, green, and blue (RGB) sensors (not shown) for detecting a range of wavelengths in the electromagnetic spectrum. In some examples, the RGB sensors may be part of a stereo camera, which includes RGB and depth sensing capabilities. In some examples, the RGB sensors may be part of a camera 120. As discussed herein, the camera 120 is used to acquire one or more images of the user, which may be used for performing skin analysis of the user. Thus, RGB information for skin analysis may be acquired via the one or more cameras 120. Example methods for performing skin analysis are described below with respect to FIGS. 3, 4, 5A, 5B, and 6 below. In some implementations, image data acquired by the one or more cameras 120 along with the datasets acquired via the UV imaging sensors 125 and/or the IR imaging sensors 123 may be used for performing skin analysis

In some embodiments, the one or more UV imaging sensors 125 and/or the one or more IR imaging sensors 123 may be used for performing skin analysis, as discussed further below. As a non-limiting example, datasets acquired via the UV imaging sensors 125 and/or the IR imaging sensors 123 may be used for skin analysis via one or more executable instructions stored in the memory 124 of the processors 122. In some examples, the datasets acquired via the UV imaging sensor 125 and IR imaging sensor 123 may be transmitted, wirelessly or via a wired connection, to one or more computing systems including processors and memory for skin analysis. Accordingly, the skin analysis system 100 may include a transceiver (not shown) for sending and/or receiving data from the one or more computing systems. When the UV imaging sensors 125 and the IR imaging sensors 123 are used for skin analysis of a subject or a part of the subject, a total field of view of the UV imaging sensors 125 and a total field of view of the IT imaging sensors 123 may be the same.

The plurality of sensors may include one or more other sensors 116 for detecting a presence and/or position of one or more objects, and/or a presence and/or position of one or more users with respect to the mirror 112. As a non-limiting example, a user may be positioned on a user side of the mirror 112 within a threshold distance from the mirror, and looking at the mirror 112. The one or more other sensors 116 may be configured to detect one or more of the presence and/or position of the user when the user is within the threshold distance. The one or more other sensors 116 may include infrared (IR) sensors 117 for user and/or object presence and/or position detection. Example sensors for object/user detection are discussed further below at FIGS. 9, 10A, and 10B. Additionally, or alternatively, other types of object detection sensors, such as time of flight sensors 119 and/or proximity sensors 121 may be used for object detection and/or human presence detection. In some embodiments, the one or more other sensors may include one or more thermopile sensors for measuring and/or estimating one or more of a skin temperature and a core body temperature of a user.

The skin analysis system further comprises one or more visible light sources 118 for illuminating one or more objects and/or one or more users with respect to the user side of the mirror. The one or more visible light sources 118 may be a plurality of light emitting diodes (LEDs) emitting wavelengths of light in the visible range. In some embodiments, the one or more visible light sources may be one or more strings of correlated color temperature (CCT) RGB LEDs configured to emit a range of wavelengths of light in the visible spectrum and may be configured to be tunable over a color temperature range that allows switching between different color temperatures via a CCT controller. As an example, the output of the CCT RGB LEDs may be automatically adjusted using a CCT controller integrated with the skin analysis system 100 to provide a desired lighting condition during image acquisition for skin analysis.

The skin analysis system further comprises one or more ultraviolet (UV) LEDs 119 for providing an UV light output in order to illuminate the subject during skin analysis of certain skin features. For example, for detection and analysis of UV-based skin features, including UV spots and sebum porphyrin, a UV-based vision system is employed to detect the UV-based skin features. The UV-based vision system includes the one or more UV LEDs for generating a UV light output within a desired intensity and wavelength range. As a non-limiting example, the one or more UV LEDs 119 may be configured to output wavelengths of light in a UVA range (e.g., 320-400 nm). The UVA light may induce skin porphyrin fluorescence in a visible range (e.g., 400 nm-700 nm), which can be imaged via the RGB camera.

In some examples, the skin analysis system 100 may further include one or more polarizing filters 130 for generating polarized light or cross-polarized light using the one or more light sources 118. Depending on one or more conditions (e.g., a type of condition analyzed, a portion of body analyzed, current lighting conditions, etc.) the visible light source may be used alone or in combination with the polarizing filters 130. As a non-limiting example, during evaluation of certain skin conditions, such as redness, brown spots etc., the skin analysis system 100 may utilize cross-polarized light to illuminate a user or a portion of the user under analysis. In one aspect, as discussed further below, the skin analysis system may automatically select appropriate light source to illuminate the user or a portion of the user depending on a type of skin analysis performed. The light illuminating the user or a portion of the user may be any of a visible light or polarized light from one or more visible light source 118, UV light from one or more UV imaging sensors or the one or more UV LEDs 119 integrated into the skin analysis system 100, or IR light from the IR imaging sensor 123 or a second IR light source (not shown) integrated into the skin analysis system 100, or any combination of light thereof. Thus, the skin analysis system may be configured to output light in a range of wavelength of the electromagnetic spectrum to illuminate the user during skin analysis.

In one example, one or more of image acquisition for skin analysis using visible light (e.g., output via CCT RGB LEDs). image acquisition for skin analysis using UV light, and image acquisition for skin analysis using IR light may be performed sequentially, in any order.

The skin analysis system 100 further includes a display 114. An example display of a skin analysis system is discussed further below at FIGS. 9, 10A, and 10B with respect to a smart mirror system.

Further, the memory 124 further includes processor-executable instructions that when executed by the processor 122, run an application on the display 114. In some implementations, the mirror 112 is of a type that is generally referred to as a one-way mirror, although it is also sometimes referred to as a two-way mirror. The mirror 112 is configured transmit a first portion of light that is incident on its surfaces to the other side of the mirror 112, and to reflect a second portion of the light that is incident on its surfaces. This may be accomplished by applying a thin layer of a partially reflective coating to a generally transparent substrate material, such that less than all of the incident light is reflected by the partially reflecting coating. The remaining light is transmitted through the mirror 112 to the other side. Similarly, some light that strikes the mirror 112 on a side opposite the side where a user is standing will be transmitted through the mirror 112, allowing the user to see that transmitted light. This partially reflective coating can generally be applied to surface of the substrate material on the display-side of the substrate material, the user-side of the substrate material, or both. Thus, the partially reflective coating can be present on the surface of one or both of the display-side and the user-side of the mirror 112. In some implementations, the partially reflective coating is made of silver. The generally transparent material can be glass, acrylic, or any other suitable material. The mirror 112 can have a rectangular shape, an oval shape, a circle shape, a square shape, a triangle shape, or any other suitable shape.

The processor 122 is communicatively coupled with the electronic display 114, the one or more UV imaging sensors 125, the one or more IR imaging sensors 123, the one or more cameras 120, the one or more other sensors 116, the one or more polarizing filters 130, and the one or more light sources 118, 119. The processor 122 may receive sensor data from each of the plurality of sensors of the skin analysis system 100, and image data from one or more cameras 120. For example, the processor 122 may receive sensor data from the one or more UV imaging sensors 125, the one or more IR imaging sensors 123, the one or more other sensors 116. Further, the processor 122 may adjust operation of one or more light sources (e.g., visible, UV, IR light sources) to adjust one or more operating parameters (e.g., intensity, duration, etc.) of light illuminating the user and/or objects at the user side of the mirror 110. As a non-limiting example, the processor 122 may adjust the one or more operating parameters of the one or more light sources according to one or more of a type of skin condition analyzed, a distance of a user from the mirror, and a body part analyzed, among other parameters.

FIG. 1B shows a block diagram of an example image processing system 121 for performing skin analysis. The image processing system 121 is the image processing system of the skin analysis system 100 at FIG. 1A. Similar components are similarly numbered, and the description of similarly number components will not be repeat for the sake of brevity. The image processing system 121 may be configured to perform skin analysis on one or more images acquired via an input device, which may be an imaging device, such as a camera 120. In one example, the skin analysis device may be a smart mirror. The smart mirror may be configured for at-home use or at a point-of-care facility, such as a clinic. An example smart mirror system is described further below with respect to FIGS. 9, 10A, and 10B.

The image processing system 121 is communicatively coupled to the camera (e.g., through a wired connection, a wireless connection, or combination thereof) and may be configured to imaging data from the camera 120. For example, responsive to initiation of skin analysis (e.g., based on indication from a user, via a user interface of the skin analysis system 100, or automatically, responsive to one or more pose, position, and timing conditions being met), the camera 120 may acquire one or more images of a body part of the user, and transmit the acquired images to the processing system 121 for further processing.

In some implementations, the processing system 121 may receive data from a storage device which stores the imaging data generated by the camera 120. In another embodiment, the processing system 121 may be disposed at a device (e.g., edge device, server, etc.) communicatively coupled to a computing system that may receive data from the plurality of sensors and/or systems, and transmit the plurality of data modalities to the device for further processing. The processing system 102 includes a processor 104, a user interface 130, which, in some aspects, may be a user input device, and display 132.

Memory 124 may store a skin detection and extraction module 152. In one example, the skin detection and extraction module 152 maybe trained to receive image data, including one or more images acquired via camera 120, and pre-process the image data to identify relevant body part for skin analysis from the one or more images and extract only skin areas for subsequent skin analysis. As one non-limiting example, responsive to a request to initiate skin analysis, a smart mirror camera may acquire one or more images of a user positioned in front of the smart mirror based on a type of skin analysis to be performed. For example, if sebum porphyrin analysis is requested (e.g., via a user indication) on facial skin, one or more acquisition parameters of the smart mirror may be adjusted based on the type of skin analysis. In particular, for the sebum porphyrin analysis one or more of a lighting condition, a number of images, and a respective pose for each image, may be adjusted so as to illuminate a user's face with UV light (from one or more UV light sources 119) and the desired number of images at the desired pose may be acquired. Responsive to acquiring one or more images, the acquired images may be input into the skin detection and extraction module 152 for detecting face, detecting skin areas from the face, and extracting skin areas. In one example, the skin detection and extraction module 152 may store a first machine learning model 154 that may be implemented for skin detection and extraction. Details of skin extraction are described with respect to FIGS. 5A and 5B.

Further, memory 124 may store a skin feature detection/segmentation module 156 that receives the extracted skin features, and identifies one or more areas based on change in tone from a baseline skin tone to detect and segment one or more skin features. Example facial skin features are provided in a table at FIG. 6, and may include one or more of sebum porphyrin, redness, acne, eczema, rosacea, UV spots, brown spots, eye bags, and scars. In one example, skin feature detection and segmentation may be performed using a transformation into a L*a*b color space from RGB color space, clustering, and histogram analysis. Details of skin feature detection are described with respect to FIGS. 5A and 5B.

Further, memory 124 may store a skin feature classification module 158 for classifying one or more skin features that were detected and extracted by the skin feature detection/segmentation module. The skin feature classification module may store a second machine learning model 160 trained to classify one or more skin features. Details of skin feature classification module are described with respect to FIGS. 5A and 5B.

In some examples, the image processing steps for skin analysis, including skin extraction, feature segmentation, and classification based on one or more machine learning algorithms may be implement at a server (e.g., server side implementation of the machine learning model(s) for skin condition evaluation, where the server is communicatively coupled to the camera and/or a processing system that is configured to receive the camera images.

Memory 124 may further store a training module, which includes instructions for training the one or more machine learning models stored in the modules 154 and 160. Training module 162 may include instructions that, when executed by processor 122, cause image processing system 121 to train one or more subnetworks in the machine learning models. Example protocols implemented by the training module 162 may include unsupervised learning techniques such as clustering techniques (e.g., hierarchical clustering, k-means clustering, mixture models, etc.), dimensionality reduction algorithms (e.g., principal component analysis) and neural network techniques (e.g., convolutional neural networks, feed-forward networks, etc.) such that the machine learning models can be trained and can classify input data that were not used for training. Further, the training module 162 may also implement supervised learning techniques, such as random forest, logistic regression, support vector machine, convoluted neural networks, such that the machine learning models can be trained on labelled datasets (e.g., datasets labelled based on clusters obtained from the unsupervised algorithm) and can generate a classification output (e.g., classification of facial features for skin extraction, classification of skin features, etc.)

Memory 124 also stores an inference module 164 that comprises instructions for testing new data with the trained machine learning model(s). Further, memory 124 may store image data 166, such as image data received from the camera 120. In some examples, the image data may include temporal data (e.g., time stamps corresponding to acquisition data and time) and user data (e.g., user identification data) so that previous skin analysis data of a user may be compared with a current skin analysis result to determine evolution of skin features over time. In some examples, the image data 166 may include a plurality of training datasets for the machine learning model(s).

Image processing system 121 may be communicatively coupled to a user interface 130. User interface 130 may be a user input device, and may comprise one or more of a touchscreen, a keyboard, a trackpad, and other devices configured to enable a user to interact with and manipulate data within the processing system 121.

Turning to FIG. 2, it shows a high-level flow chart illustrating a method 200 for performing skin analysis for a user. The method 200 may be executed by a processor, such as the processor 122 of the skin analysis system 100, according to instructions stored in a non-transitory memory, such as the memory 124. The method 200, and other methods herein will be described with respect to the skin analysis system 100, although the methods may be implemented by other systems without departing from the scope of the disclosure.

The method 200 may be initiated in response to acquiring input from one or more of a camera, an object detection sensor, and user. In one example, responsive to detecting, via an object detection sensor (e.g., sensors 116), an object within a threshold distance from a mirror, such as mirror 110, the skin analysis system may activate a camera (e.g., camera 120) and receive input from the camera, and/or may activate a user interface (e.g., display 114) of the skin analysis system to acquire user input. In another example, the user may enter a request via the user interface of the skin analysis system, and the method 200 may be executed in response to the user input.

At 202, method 200 includes determining if skin analysis is performed. In one example, a user may request skin analysis via the user interface. In some examples, the camera may recognize the user, and access the user's preferred setting from the memory of the skin analysis system, which may include an indication as to whether skin analysis may be performed. Additionally, or alternatively, the camera may recognize a pose and a position of the user with respect to the mirror, which may provide an indication as to whether skin analysis is desired. In still another example, the skin analysis system, upon detecting user within a threshold distance from the mirror, may automatically determine that skin analysis is desired.

If it is determined that the skin analysis is not desired, the method 200 proceeds to 222, wherein one or more sensors related to skin analysis, such as camera, IR imaging sensor 123 and UV imaging sensor 125 may not be activated, and/or lighting systems, such as UV LED may not be operated or powered ON. The other object/position detections sensors may be activated and/or may remain active.

If skin analysis is desired, the method 200 proceeds to 204. At 204, the method 200 includes determining a type of skin analysis. In one example, determining the type of skin analysis may include determining if analysis of one or more selected conditions (e.g., redness, sore, UV spot, burn, cut, etc.), one or more features (e.g., wrinkle, eye bag, etc.) and/or one or more regions (e.g., face, forehead, nose, area below the eyes, cheek, etc.) is desired. The type of skin analysis may be determined based on a current user input and/or stored user preference, for example. In one aspect, the type of skin analysis may be determined automatically based on a previous skin analysis result of the user. In some examples, the skin analysis system may suggest or recommend one or more types of skin analysis based on a preliminary skin analysis of the user. In still another example, a selected type skin analysis (e.g., detection of cut) may be automatically initiated after and/or during an activity detected by the skin analysis system (e.g., shaving).

Next, at 206, the method 200 includes activating one or more of camera, UV imaging sensor, and IR imaging sensor. Further, the camera and object/position detection sensors may remain active. In one embodiment, one or more sensors may be selectively activated based on the type of skin analysis (as indicated at 208). As a non-limiting example, if the type of analysis includes a detection, estimation, and/or measurement of porphyrins on the face, one or more UV light source may be activated and utilized for performing the selected type of skin analysis. As another non-limiting example, if redness analysis is desired, visible light source may be activated to illuminate the user during image acquisition. A non-limiting list of example conditions, features, and regions, and the corresponding light source (and therefore, sensor types) for skin analysis is shown and described with respect to FIG. 6.

In another embodiment, a plurality of sensor types may be activated to perform a more comprehensive skin analysis. As a non-limiting example, one or more UV imaging sensors, one or more IR imaging sensors, and/or one or more RGB sensors may be activated. Further, one or more visible light sources and its associated polarizing filters may be activated to generate a skin profile for the user. The skin profile may be generated with respect to a portion of the user's body (e.g., entire face, hand, etc.) and/or with respect to a region within the portion (e.g., forehead, thumb, etc.) in the field of view of the mirror of the skin analysis system. In one embodiment, activating the plurality of sensor types may include adjusting a corresponding field of view of each sensor type according to a camera field of view and/or the field of view of the mirror. In another embodiment, activating the plurality of sensor types may include supplying electrical power to one or more imaging sensors.

Next, at 212, the method 200 includes locating a user position with respect to the mirror. For example, one or more proximity and/or object detection sensors may determine a position of the user with respect to the mirror. In some examples, user position may be determined with respect to the one or more light sources and/or the imaging sensors disposed within the mirror. In another example, determining the position may further include determining the user or the body part of the user for which skin analysis is to be performed is within the corresponding field of view of the one or more sensors required for skin analysis and the camera.

Next, at 214, the method 200 includes operating the one or more UV imaging sensors, the one or more IR imaging sensors, and/or visible light sources (and its associated filter if polarized light is required based on a current lighting on the user and/or type of skin condition) to direct one or more of UV, visible, and IR light to illuminate the user or a part of the user. In one embodiment, using input from the camera, a body part (e.g., face) of the user may be recognized and a desired portion of the body part (e.g., forehead) may be identified and illuminated. Said another way, the corresponding field of view of the sensors may cover the desired portion of the body part. In some examples, as will be discussed further below, an entire body part (e.g., face) is illuminated and one or more images of the entire body part may be acquired. The desired skin portion may be subsequently extracted, based on machine learning algorithms, for skin analysis (e.g., feature detection and quantification)

Next, at 216, the method includes acquiring imaging data via the one or more camera, UV imaging sensors, the IR imaging sensors, and/or camera. In one example, imaging data may be acquired via the one or more cameras (e.g., RGB cameras), and the skin analysis may be performed on the one or more images acquired by the one or more camera(s). However, depending on the type of skin analysis a lighting condition for illuminating the user may be adjusted. For example, if sebum porphyrin analysis is desired, UV light source is activated to illuminate the user, and the fluorescence output from the user's skin that occurs in the visible region is detected and captured by the one or more RGB cameras.

Further, in one example, a corresponding intensity of light (from each light source (e.g., UV, IR, visible)) transmitted and illuminating the user may be adjusted according to a distance of the user or the portion of the user under illumination with respect to the mirror. As a non-limiting example, as a distance of the user decreases, the intensity of light illuminating the user is decreased. Further, in some examples, if the distance of the user is greater than a threshold distance, an indication may be provided (via the display, or speaker coupled to the skin analysis system) to guide the user to remain within the threshold distance.

Further, in another example, one or more of a corresponding intensity, duration, and/or wavelength of light (from each light source) is adjusted based on the type of feature analyzed.

Further, in another example, when a time-related analysis is performed to evaluate a skin condition over time, illumination and acquisition parameters may be consistent at each time point.

Upon acquiring imaging data for skin analysis, the acquired data is analyzed by the skin analysis system, and one or more results of the skin analysis is stored and/or displayed via the display to the user. An example method for analyzing the sensor and/or camera data will be described below with respect to FIGS. 3, 5A, and 5B.

Referring to FIG. 3, a high-level flow chart illustrating a method 300 for analyzing sensor data acquired via a skin analysis system, such as the skin analysis system 100 is shown. The method 300 may be executed by a processor, such as the processor 122 of the skin analysis system 100, according to instructions stored in non-transitory memory, such as the memory 124. In some examples, the method 300 may be carried out by a computing system communicatively coupled to the processor. The method 300 describes example analysis when two or more types of sensors (e.g., UV, IR, camera) are used to generate an electromagnetic spectrum profile for a user.

At 302, method includes acquiring one or more imaging datasets via one or more UV sensors (e.g., UV imaging sensors 125), one or more IR sensors (e.g., IR imaging sensors 123), and camera (e.g., one or more camera 120) of the skin analysis system. Details of acquiring imaging data is discussed above at FIG. 2.

At 304, method 300 includes pre-processing each dataset, including dataset from each imaging sensors and camera. Pre-processing each dataset may include filtering to remove noise, glare, etc.

At 306, upon pre-processing, method 300 includes processing each dataset to identify one or more regions and features within respective images of each dataset. For example, an input image may be generated from each dataset. Each input image may be segmented using different image processing techniques, such as but not limited to, edge detection technique, boundary detection technique, thresholding technique, clustering technique, compression based technique, histogram based technique or a combination thereof. The segmentation may be used to identify one or more regions and features within respective input images of each dataset.

In one implementation, the one or more regions and features may be based on user facial geometry (if skin analysis of face is performed) or other body geometry (depending on the body part analyzed). Accordingly, in one embodiment, when facial skin analysis is performed, the one or more regions may include facial regions such as a forehead region, eye region, temple region, cheek region, nose region, mouth region, ear region etc. Each region may be further divided into sub-regions (e.g., right and left sub-regions for each region). In another embodiment, the one or more regions may delineate specific facial features such as eye, eyebrows, mouth, nose, etc.

In another implementation, one or more region and features may include general regions and features based on facial geometry, and may further include skin condition regions and features based on a variety of skin conditions. The variety of skin conditions may be based on the portion of the body analyzed, for example. As an example, for facial skin analysis, a first set of facial skin conditions may be analyzed (e.g., skin conditions that are associated with facial skin, such as under-eye bag, dark circle, etc.), and therefore, the regions and features identified in each data set may include facial regions and features, as well as features that are characteristic of the first set of facial skin conditions (including, in some examples, current known facial skin condition for the user).

In one example, as indicated at 308, one or more machine learning algorithms may be used to identify the one or more regions and features. The one or more machine learning algorithms may be a deep learning algorithm implemented by one or more convoluted neural networks that are trained with training datasets comprising features corresponding to various skin conditions.

In this way, each of imaging dataset (e.g., a UV imaging dataset acquired via the one or more UV imaging sensors, an IR imaging dataset acquired via the one or more IR imaging sensors, and a camera imaging dataset acquired via the camera), is processed to identify one or more regions and features in the respective imaging datasets. Said another way, each imaging dataset is divided into a plurality of groups based on the identified regions and/or features. As a non-limiting example, when UV, RGB, and IR sensors are used, for a given region (e.g., forehead) of a body part (e.g., face) of the user, three datasets are generated. As a result, a corresponding skin analysis map is generated for each sensor dataset for the body part under analysis.

Next, at 310, the one or more imaging datasets are combined so as to generate an electromagnetic spectrum profile for each of the one or more region and features. The electromagnetic spectrum profile is a combined skin profile including data acquired via two or more sensors for each of the one or more regions and features (identified at 306), the two or more sensors capturing skin response to various wavelengths comprising ultraviolet, visible, and/or infrared wavelengths across the electromagnetic spectrum. The electromagnetic spectrum profile may include quality and quantification data regarding skin response to different light sources. In this way, for each of the one or more regions and features detected, the electromagnetic spectrum profile that provides quality and/or quantification data is generated by combining the imaging datasets. Said another way, the skin analysis maps (generated at 306) are combined with imaging data from each of the sensors.

As a non-limiting example, when the UV, RGB, and IR sensors are used for analysis, the UV imaging dataset may include UV response data, the IR imaging dataset may include IR response data, and the visible light imaging dataset may include visible light response data. It will be appreciated that camera data may be used to acquire RGB data. The UV, RGB, and IR response data are combined with the skin analysis map (details of generating a 3D map is discussed at FIG. 5A, with respect to step 508) to generate the electromagnetic spectrum profile, wherein each of the one or more regions and features of the skin analysis map includes UV, RGB, and IR response data. In one example, each of the one or more regions and features may include a reference to corresponding response features acquired via one or more sensors. For example, each of the one or more regions may be a pixel group (comprising one or more pixels) or a voxel group (comprising one or more voxels) including references to UV and visible light response features when UV and RGB sensors are used. Similarly, each of the one or more regions may be a pixel group or a voxel group including references to visible light response and IR response features when RGB and IR sensors are used.

Next, at 312, a current skin profile is generated based on the electromagnetic skin profile. In one example, the current skin profile may be the electromagnetic spectrum profile for a desired region and/or feature. In another example, the current skin profile may be the electromagnetic spectrum profile for a combination of regions and/or features.

In some implementations, additionally or alternatively, a temporal skin profile (as indicated at 314) may be generated based on current skin profiles acquired at different time points. The temporal skin profile may be used to monitor skin condition changes over time. The temporal skin profile may be generated for the body part, the one or more regions and features in the skin analysis map, or for specific skin condition features, or any combination thereof.

In one example, the skin analysis map and the sensor response data for each region and/or feature in the map may be used to track progress or change of a given skin condition over time. Using the skin analysis map, a location of the given skin condition may be automatically located and the progression or change of the skin condition may be automatically monitored. As a result, the user is not required to identify or track the region/feature each time. In this way, accuracy of skin analysis is greatly improved.

Next, at 316, the method 300 includes outputting a result (or assessment) of the skin analysis according to the current and/or temporal skin profile. The result may be displayed to the user via the display of the skin analysis system, stored in the non-transitory memory, and/or transmitted to a receiver (e.g., clinician, care provider, etc.) for further analysis and/or evaluation. In some embodiments, the result (or assessment) may be displayed via augmented reality, wherein the results are overlaid on a reflection of the user's face in the mirror and/or a camera image of the user's face displayed via the display portion of the mirror.

In this way a skin analysis map of a body part (e.g., a 2D or a 3D face map) is generated, wherein each pixel group or voxel group in the map includes a reference to corresponding response features identified using UV light and/or visible light (and/or IR light). Thus, skin features identified using visible light are mapped onto the 2D or 3D face map, and the skin features identified using UV light are also mapped onto the 2D or 3D face map. Thus, each region in the 2D or 3D face map includes a reference to UV light features as well as visible light features. During subsequent analysis over time, in addition to providing a comprehensive skin profile analysis, the 2D or 3D face map may be used to track evolution of any given skin feature over time.

FIG. 4 shows a high-level flow chart illustrating an example method 400 for acquiring imaging data for skin analysis, according to another embodiment of the disclosure. The inventors herein have identified that positioning of the user may be adjusted to obtain a more accurate and comprehensive analysis, and that the position of the user may be different for different skin conditions. Accordingly, method 400 may be implemented for evaluation of one or more skin conditions for which, for each sensor, imaging data is acquired for different poses of the user in order to obtain a more accurate and comprehensive analysis of the skin condition. It will be appreciated that acquiring imaging data at different poses may also be used for generating the electromagnetic spectrum profile as discussed at FIG. 3 and is within the scope of the disclosure.

At 402, the method 400 includes determining if one or more additional pose data is required for a current skin analysis. In one example, the requirement for one or more additional pose data is determined according to one or more skin conditions analyzed. In another example, the requirement for additional pose data is determined according to use input. If additional pose data is required, method 400 proceeds to 404; otherwise, the method 400 proceeds to 418 to process acquired imaging datasets to generate current and/or temporal skin profile as discussed at FIG. 3.

At 404, method 400 includes providing an indication to the user to move to a desired pose. The indication may be one or more of a visual and voice indication including instructions for user positioning.

Next, at 406, method 400 includes determining if the desired pose is achieved. In one example, the camera may acquire one or more images, and the images may be evaluated to determine if the desired pose is achieved (e.g., according to an outline, features visible, etc.).

If the user is not at the desired pose, the method 400 proceeds to 416. At 416, the user may be given one or more additional indication regarding adjustments to arrive at the desired pose. The method 400 then returns to 406 to continue pose evaluation.

If the answer at 406 is YES, method 400 proceeds to 408 to acquire imaging datasets using one or more imaging sensors as discussed at 216. Further, one or more of an intensity and duration of the incident light may be adjusted according to a distance of the user from the mirror, as discussed at 218. Furthermore, one or more of the intensity, duration, and wavelength of the incident light may be adjusted according to a type of feature and/or skin condition analyzed, as discussed at 220.

Next, at 414, the method 400 includes integrating different pose data to generate a combined pose imaging data set for each sensor, which are then processed as discussed FIG. 3.

FIG. 5A shows a high level flow chart illustrating an example method 500 for performing skin analysis, according to an embodiment. In particular, the method 500 may be implemented based on instructions stored in non-transitory memory of a processing system of a skin analysis system, such as the skin analysis system 100 of FIG. 1, a server in communication with the processing system, an edge device connected to the processing system, a cloud in communication with the processing system, or any combination thereof. The method 500 will be described with respect to FIG. 1; however, it will be appreciated that the method 500 may be implemented using similar systems without departing from the scope of the disclosure.

At 502, the method 500 includes acquiring one or more images of a user. In one example, the one or more images of the user may be acquired via a camera, such as camera 120, integrated with the skin analysis system. The camera may be an RGB camera, for example. The one or more images of the user includes images of the body part for which skin analysis is desired. Acquiring one or more images of the user includes, at 504, acquiring different views or angles of the user. In particular, different views or different angles of the user's body part is imaged using the camera in order to generate a three dimensional map of the user's body part for which skin analysis is desired. In one example, when facial skin analysis is desired, one or more images of the user's face is acquired. For example, a front view, a right side view, and a left side view of the user's face may be imaged via the camera. The examples herein will be primarily discussed with respect to skin analysis on face, however, it will be appreciated that the systems and methods described herein may be implemented to perform skin analysis of any body part (e.g., hand(s), leg(s), elbow(s), back, etc.), and are within the scope of the disclosure.

An example method of acquiring one or more user images is described above with respect to FIG. 2. Briefly, one or more parameters of the skin analysis system may be adjusted and/or selected based on the type of skin analysis. The one or more parameters may include one or more of a light source for illuminating a desired body part of the user for skin analysis (e.g., UV light source, CCT RGB light source), intensity of the light source, one or more filters through which the light from the light source passes through before illuminating the desired body part of the user (e.g., diffuser, cross-polarized filter, parallel-polarized filter, etc.), a field of view of the camera, a number of views to be imaged with the camera. As a non-limiting example, if redness is to be analyzed, the light source may be configured to output daylight RGB and a cross-polarized filter may be selected. Further, in some embodiments, the entire face may be imaged, and during image processing, the relevant portions of the facial skin for redness analysis, such as facial skin of cheeks and nose, may be extracted as discussed further below. In some examples, additionally a field of view of the camera may be adjusted to image a portion of the face including cheeks and nose (but not forehead), and subsequently, the image is processed to extract the relevant skin of cheeks and nose. Further, the camera may be further configured to acquire images of a frontal view and 2 lateral views. Further still, user may indicate one or more additional regions for analysis, and the image may be acquired and processed accordingly.

In some examples, the user may indicate, via a user interface, the type of analysis that is desired. Further, in some examples, more than one analysis may be performed. For example, analysis of sebum distribution and redness may be desired. Accordingly, a first set of images may be acquired with UV light and no polarizing filters for sebum distribution analysis and a second set of images may be acquired with daylight RGB and polarized filter for redness analysis.

In some examples, a comprehensive skin analysis may be desired, wherein analysis of all skin features is performed. In such examples, two or more sets of images may be acquired, each under different lighting and camera settings corresponding to the type of skin analysis. Further, as discussed above with respect to FIGS. 2 and 3, a user may be indicated (e.g., voice and/or visual indications), via a user interface of the skin analysis system, to adjust and/or hold one or more of position of the body part under analysis (front view, lateral view, etc.), a distance from the skin analysis system, and adjust external lighting (e.g., for imaging of UV-induced fluorescence of sebum). In some examples, the skin analysis controller may communicate with a controller of a smart lighting system to adjust (e.g., turn off) lighting and/or a controller of a smart window shades system (e.g., to darken blinds) in an operating environment of the skin analysis system to automatically adjust the external lighting based on the type of skin analysis.

Next, upon acquiring one or more images of the user, the method 500 proceeds to 506. At 506, the method 500 includes generating a composite image from the one or more images. In some examples, when more than one set of images are acquired, for example, for evaluating different skin features under different lighting conditions, a composite image may be generated for each set of images. For example, a first set of images may be acquired for analysis of a first skin feature under a first lighting condition (e.g., with UV light and no polarizing filters for sebum distribution analysis) and a second set of images may be acquired for analysis of a second skin feature (e.g., with daylight RGB and polarized filter for redness analysis) under a second lighting condition. Using the first set of images, a first composite image may be generated, and using the second set of images, a second composite image may be generated. Each of the different composite images may then be analyzed as discussed further below.

Further, at 508, the composite image is processed to generate a three dimensional map (3D) of the body part is generated. Particularly, in order to be able to adapt to face geometry, a 3D model-wise analysis of the skin feature based on the 3D map is performed. As discussed above, one or more images of the body part under analysis are acquired at different orientations (steps 502 and 504). A 3D model of the body part (e.g., face, hand, etc.) is then generated using one or more images. Further, a 3D map of the 3D model is generated. The 3D map may be a 3D mesh (e.g., a face mesh) comprising a set of vertices in a 3D coordinate space, and edges connecting specified vertices. For example, the processing system of the skin analysis system may include map generator module to generate the mesh. For instance, responsive to receiving an images (e.g., the composite image from the one or more images, or a single image acquired via the camera), the map generator generates a mesh for the one or more body parts in the image. The map generator can execute, for example, Delaney Triangulation, linear interpolation, and/or cubic interpolation techniques to generate the mesh. For instance, Delaney Triangulation can be performed to define triangle indices for the vertices, such as the landmark points. In one or more implementations, the map generator can perform linear interpolation to determine landmark points of the body part. For example, a number of landmarks of the body part may be estimated (e.g., facial landmarks such as eyes, nose, mouth, etc.,) to estimate face geometry and/or face contours, using the 3D map (e.g., a face map). Further, the 3D map is used to identify relevant skin portions for analysis so that the relevant skin portions may be extracted for subsequent analysis. In this way, speed and accuracy of the skin condition analysis is improved.

Further, the mesh is used as a map for correlating facial geometry and contours from different images of a user taken at different time points, which in turn enables tracking of skin analysis over time. Further, any two different time points are separated by a period, which may be minutes, hours, days, months, years. In this way, the mesh generated from the camera images allows changes in skin features to be monitored over time. As such, a user may determine whether a given skin condition (e.g., eczema) is changing (e.g., improving, worsening, degree of change) or remaining the same over time. Further, by monitoring the temporal changes to a skin condition, the user may determine whether a skin treatment has been effective. Temporal evolution of skin features will be discussed further below with respect to FIG. 5B.

Next, at 510, the method 500 includes detecting the desired body part from the composite image. In some embodiments, a single image may be used, and the desired body part may be detected from the single image. In some embodiments, using one or more images, the desired body part is projected in two dimensions. As a non-limiting example, if skin analysis is performed on facial skin, the method includes detecting the face from the one or more images, where the one or more images include one or more of a composite image, a two dimensional projection image generated from the composite image, a two dimensional projection image generated from a three dimensional image, and a single image acquired via the camera. Detecting the face includes finding an area in the image where the face of the user is. Similarly, body part detection includes detecting an area where the body part is present in the image. Various machine learning techniques may be used for detecting a desired body part in the image. For example, feature-based detection methods may be used. Alternatively, image-based detection methods may be used. Example feature-based detection methods may include one or more of an active shape model (e.g., deformable template model, deformable part model, snakes, point distribution model, etc.), a low level analysis (e.g., skin color base face detection model, edge-based face detection model, etc.), and feature analysis models (e.g., feature searching models). Example image-based detection methods include one or more of a neural network model (e.g., feed forward neural network, retinal connected neural network, back propagation neural network, convolutional neural network, polynomial neural network, rotation invariant neural network, back propagation neural network, radial basis function neural network, etc.), linear subspace methods (e.g., eigenface, fisherfaces, tensorfaces, probabilistic eigenspace, etc.), and statistical approaches (principal component analysis, support vector machine (SVM), discrete cosine transform, independent component analysis, locality preserving projection etc.). As a non-limiting example, a semantic segmentation mask technique may be used to identify the body part from the image. For instance, a facial mask detection using semantic segmentation may be used. As another non-limiting example, for facial skin analysis, features may be extracted using PCA or other feature extraction algorithms, and a SVM model may be used to classify between face areas and non-face areas.

Further, at 510, the method 500 includes extracting skin areas from the detected body part. Extracting skin areas include performing image segmentation on the detected body part image to locate and identify boundaries of different features within the detected body part image. That is, one or more features of the detected body part may be segmented and various features of the body part may be classified including skin areas. For example, facial features (e.g., eyes, nose, lips, eyebrows, skin) may be segmented and classified. After segmentation, the skin areas are extracted and used for subsequent skin analysis, and the remaining areas corresponding to one or more remaining features are not subject to skin analysis. An example processed image including the relevant skin areas after extraction for analysis of redness is shown at FIG. 7A. Turning to FIG. 7A, facial image segmentation generates an output including one or more boundaries according to respective contours of the one or more facial features. In this example, the facial image is segmented to identify and classify eyes, nose, lips, and skin. Further, the eyes, nose, and lips are masked and not subject to skin analysis, and only the skin areas are extracted and subject to analysis downstream.

In some examples, the detection of body part and extraction of skin areas may be combined and performing using one or more machine learning models indicated above.

Returning to FIG. 5A, upon extracting the skin areas for analysis, the method 500 proceeds to 512. At 512, the method 500 includes encoding the extracted image onto a multi-dimensional array. In one example, the method 500 includes encoding the image with extracted skin areas on to a four dimensional matrix. The four dimensional matrix includes two dimensions for space vector (X, Y), and two dimensions for the AB CIE 1976 color space. Thus, the exacted image is transformed into a four dimensional XYAB domain. The AB CIE 1976 color space is also referred to as L*a*b* color space, where L* indicates lightness, and a* and b* are chromaticity coordinates. In particular, a* and b* are color directions, where +a* is the red axis, −a* is the green axis, +b* is the yellow axis and −b* is the blue axis. In this way, skin analysis is performed based on LAB color space that is more perceptually linear, where a change in an amount of a color value (e.g., redness) produces a change directly proportional to visual importance. Accordingly, accuracy of skin analysis is improved.

Next, at 514, the method 500 includes normalizing the four dimensional matrix dataset. In particular, normalizing the four dimensional matric dataset include normalizing based on weights to give more importance to color than space. Accordingly, normalizing the four dimensional matric dataset includes adjusting weights such that color is weighted more than space.

Next, at 516, the method 500 includes clustering the normalized dataset into N number of clusters. The clustering of colors is performed according to a clustering algorithm. In one example, K-means clustering may be used. Further, a number of clusters for the K-means clustering may be determined according to an elbow method, for example. Other unsupervised learning methods, such as Fuzzy C-means clustering, Self-Organizing Map (SOM), etc., may be used and are within the scope of the disclosure. An example skin clusterization visualization after the XYAB domain transformation is shown at FIG. 7B. In particular, FIG. 7B shows an enlarged portion of FIG. 7A and depicts a plurality of redness clusters after XYAB domain transformation and clustering. Dominant redness areas are depicted at 703 and 705.

Next, at 518, the method 500 includes reducing dimension of the clustered colors to encode color tones into unique scalars. In particular, a dimension reduction method is applied to the clustered color data and transform the color tones into unique scalars. As one non-limiting example, principal component analysis is performed on the clustered color data to encode color tones into unique scalars.

Next, at 520, the method 500 includes determining tone distribution and outliers of colors to segment various skin features on the skin. In one example, a histogram analysis is performed after PCA dimension reduction with peak analysis to detect outliers. As a non-limiting example, for redness analysis of skin, a redness histogram analysis is performed after the PCA domain reduction, with peak analysis to detect redness outliers and segment various redness areas. An example redness histogram analysis after the PCA domain reduction, with peak analysis to detect redness outliers is shown at FIG. 7C. In particular, FIG. 7C shows histogram analysis of the image portion shown in FIG. 7C. Further, an example of redness detection is shown at FIG. 7D. In particular, areas 704 and 706 show segmented areas corresponding to redness areas 703 and 705 respectively shown at FIG. 7C. The areas 704 and 706 are further processed to quantify redness based on distance to baseline skin tone. Details of an example of redness quantification is discussed below at FIGS. 8A-8E with respect to another portion of the image shown in FIG. 7A.

In some examples, a plurality of skin features may be segmented using clustering, PCA, and histogram analysis. As a non-limiting example, for facial skin analysis, the plurality of skin features may include, but not limited to one or more of sebum porphyrin, redness, UV spots, brown spots, eye bags, and scars. Based on differences in one or more of color, tones, and hues, with respect to a baseline skin tone of the user, a plurality of skin features are segmented. In this way, a plurality of skin features are detected based on segmentation. Further, in some examples, a number of areas for a particular skin feature (e.g., a number of redness areas) among the plurality of skin features may be detected based on the segmented features. In some examples, a number of areas of each skin feature may be detected based on the segmented features.

Thus, for a facial skin analysis, a first segmentation is performed on the image (e.g., composite image or acquired image) to segment facial features, where the facial features include one or more of eyes, nose, lips, eyebrows, and skin. Based on the first segmentation, only skin areas are extracted. Using the extracted skin areas, a second segmentation based on clustering, PCA, and histogram analysis is performed on the extracted skin areas to detect one or more skin features of face, where the one or more skin features include one or more of sebum porphyrin, redness, acne, eczema, rosacea, UV spots, brown spots, eye bags, and scars. An example of quantification of redness, after redness detection is described at FIGS. 8A-8E.

Turning to FIGS. 8A-8E, it shows a set of images depicting visualization of redness quantification, according to an embodiment of the disclosure. FIG. 8A shows example redness clusters of an example skin portion of FIG. 7A. In particular, a chin portion of the image shown in FIG. 7A is shown in FIG. 8A. Further, FIG. 8A shows example redness clusters at 802 and 804. As mentioned above, FIG. 8A shows visualization of redness clusters after transformation into XYAB domain. The clustering approach enables identification of dominant colors.

FIG. 8B shows the dominant colors in the image portion of FIG. 8A, the dominant colors being colors that are represented (e.g., greater spatial representation) to a greater extent than other colors. As depicted the redness colors corresponding to redness clusters 802 and 804 are indicated as dominant colors in addition to baseline skin tone.

Next, FIG. 8C shows an output of a superposition strategy to detect redness areas 804 and 806. In particular, the redness areas are superposed with masks. The superposed areas are depicted by 808 and 810.

Next, a filtering technique is applied to the superposed image for denoising purposes. A filtered image in shown at FIG. 8D with the corresponding redness areas indicated by 810 and 812. Finally, a final distance of the redness areas with to baseline skin color features are determined to quantify the redness features. FIG. 8E shows the final color distance of the redness areas depicted by 814 and 816 (corresponding to redness clusters 802 and 804). In one example, the Euclidean distance metric is used. Other distance metrics, such as Mahalanobis distance and Manhattan distance may be used and are within the scope of the disclosure.

Referring next, at 524, the method 500 includes classifying a plurality of skin features using a trained machine learning algorithm. In one example, classifying the plurality of skin features includes performing a multi-class classification to identify each of the plurality of skin features. For example, for facial skin analysis, multi-class classification may be performed to identify one or more of sebum porphyrin, acne, eczema, rosacea, UV spots, brown spots, eye bags, and scars. In some examples, a neural network model may be trained to identify a skin feature. In some examples, the neural network model may be co-trained on a plurality of datasets corresponding to the plurality of skin features to output a multi-class classification result. In some other examples, a skin analysis model may comprise a plurality of neural network models, each trained to output a classification of a different skin feature. The neural network model for skin feature classification may be a convoluted neural network model, a feed forward network, etc.

In one example, a supervised learning method may be employed to train the neural network for classification of skin features. In some examples, the training and validation datasets may be generated using segmentation performed as discussed at steps 502 to 522, and annotated for supervised learning approaches.

Next, at 528, the method 500 includes performing quantification of one or more classified skin features. In some examples, the method 500 includes performing quantification of the segmented skin features at step 520. In particular, quantification may be performed on the output of step 522.

Performing quantification of the one or more skin features (e.g., classified skin features and/or segmented skin features) includes, at 530, performing one or more of a local quantification, a global quantification, and feature evolution determination. Details of the quantification is discussed below at FIG. 5B. Next, at 532, the quantification results are output, via a user interface of a smart device (e.g., a smart mirror device).

Referring now to FIG. 5B, it shows a high level flow chart of an example method 550 for quantifying one or more skin features. The quantification described below in method 550 may be performed on classified skin features (output of step 524) and/or segmented skin features (output of step 522).

At 551, the method 550 includes determining surface occupation of each feature. In one example, determining surface occupation includes determining an individual size of each feature (at 552) for local aspect analysis. Accordingly, the local aspect analysis includes determining one or more of an individual feature size and a degree of intensity of coloration change from a reference skin color of the user (e.g., change with respect to a normal skin color and tone surrounding the skin feature under analysis). In some examples, additionally, or alternatively, at 554, the method 550 includes determining overall coverage relative to the body part analyzed, for global aspect analysis. Thus, the global aspect analysis includes determining one or more of an occupation ratio for each feature (e.g., a percentage of skin occupied by the feature), a total occupation ratio for a number of features (e.g., a percentage of skin occupied by a number of features detected and/or analyzed) and a relative feature ratio (e.g., a percentage of a given feature compared to total number of features identified).

Next, at 556, the method 550 includes outputting the local and/or global quantification result. In one example, the local and/or global quantification results are displayed on a display portion of a user interface of the device (e.g., smart mirror) performing the image acquisition and skin analysis. As an example, the quantification results may be displayed as an overlay over one or more of an image, a composite image, and a three dimensional image acquired for skin analysis. Further, the quantification result may include a type of skin feature (e.g., eczema, redness), a degree of severity of the skin feature (e.g., a degree of eczema, a degree of redness), the areas of the skin covered by each feature (e.g., a segmented outline of an area of redness), global aspects (e.g., a percentage of total skin surface area occupied by redness), local aspects (e.g., size, volume, shape, color, specific location(s) within the body parts etc.), and evolution aspects (e.g., change is local and/or global aspects over time). The quantification of evolution aspects will be described below at steps 560-564.

Turning back to the local and/or global quantification results, in one example, one or more skin features may be segmented and the segmentation indication (e.g., indications of a boundary of the skin feature) may be displayed on the display portion. Further, the local features, including feature size, volume, a number of features, location of the features and occupancy of the features in each of these locations (e.g., present on right cheek only, percentage identified in right cheek, percentage identified in left cheek, etc.), may be determined and displayed. Additionally or alternatively, the quantification results may be displayed as a graph, tabular output, or any other suitable visual indications. Further still, in some embodiments, other forms of indication, such as voice indication, may be additionally or alternatively provided.

In some examples, additionally, or alternatively, the quantification result is transmitted to a mobile device (e.g., smart phone, tablet, etc.) communicatively coupled to the device (that is, the smart mirror). In some examples, additionally or alternatively, the quantification result is transmitted to a database that may be accessed by an authenticated user. In some examples, when the skin analysis is performed via a computing device communicatively coupled to the smart mirror, the quantification result is transmitted to one or more of the smart device, mobile device, and any other connected device communicatively coupled to the smart mirror. Further, the quantification result is stored in a database of the computing device, which can be accessed by an authenticated user.

Referring to 560, the method 550 includes locating each feature within the 3D map of the body part (e.g., 3D map generated at step 508) in order to determine an evolution of one or more skin features over time (e.g., evolution of redness, evolution of eczema, evolution of sebaceous features, evolution of eye bags, evolution of brown spots, evolution of any discolorations, etc.). For instance, the evolution of skin features is used to manage treatment one or more skin features, evaluate effectiveness of a make-up regimen, etc. For example, by monitoring the evolution of a facial skin feature (e.g., acne, scars), the user may determine whether the skin feature is changing in response to a treatment and/or monitor natural evolution of the skin feature over time. Upon locating each skin feature (e.g., acne, wrinkles, redness, eye bag, etc.), the respective coordinates of each skin features on the 3D map is stored.

Further, the evolution of skin features may be used to determine a trend in evolution of one or more features. For example, the trend in evolution of the skin features may be monitored to determine whether one or more skin features appear at a certain timing and/or interval (e.g., appearance of skin features coinciding with a menstruation cycle), whether one or more skin features appear due to seasonal skin changes (e.g., increase in yellowish hue of skin due to increase in pollen during spring time, etc.), is a new skin condition, and/or is a symptom of a health condition (e.g., psoriasis, gastrointestinal health condition, etc.), among other trends.

Further still, in some example, in addition to monitoring one or more features, the skin tone, hue, and/or color itself may be monitored over time to identify changes in skin conditions. For example, increase in paleness of skin over time may be monitored to determine a probability of vitamin deficiency (e.g., vitamin B12). Likewise, effectiveness of a skin treatment for the skin (in addition to or alternative to treating the features) may be monitored over time to evaluate a health of the skin based on skin tone, hue, and/or color changes.

Accordingly, after locating each skin feature within the 3D map of the body part (e.g., 3D face map), the method 550 proceed to 562. At 562, the method 550 includes determining a change in one or more skin features over time. The change in one or more skin feature may be determined based on one or more previous acquisitions, and the corresponding local and/or global feature analysis. For example, based on the location of a skin feature, for example, a redness feature, the method may determine whether the current redness feature has increased, decreased, or remains the same since last acquisition and analysis. The change in a skin feature includes one or more of a change in size (e.g., change in area, volume), a change in severity, and a change in a distribution of a given feature over time (e.g., a change in facial sebum distribution over time), and change in color over time (that is, if a feature at a given location coordinates in a 3D map of the body part under analysis shows a change in color).

Next, at 564, the method 500 includes outputting one or more of a local evolution result and a global evolution result for one or more skin features (classified and/or segmented), which includes outputting for one or more skin features, a change in feature characteristics (e.g., size, color shift, distribution, etc.) over a plurality of different time points within a duration of time. Further, the time points, time intervals between time points, duration of time, and other temporal conditions for evolution may be selected automatically based on the type of feature analyzed, or selected based on user input.

Upon outputting the local and/or global skin analysis results and/or the corresponding evolution results, the method end.

In this way, the methods and systems described herein may be used to determine local aspects of one or more skin features, such as a size of each spot (redness, sebum, wrinkles etc.), and further, detect if the size changes through time (e.g., does these spots increase or decrease in size, and/or do they change in color). Further, the methods and systems described herein may be used to determine global aspects. For example, the global aspects may be used to provide to the user an occupation ratio (e.g., how much of the face skin % is occupied by skin features). Further, one the identified skin features, one or more metrics about the evolution over time (increase, decrease, color shift, etc.) is provided.

Further, the methods and systems described herein allows a skin feature to be evaluated with respect to surrounding skin areas (that is, normal skin areas). That is, the methods and systems described herein provide relative skin color and tonal assessment. Thus, the methods and systems described herein may be adapted to various human skin tones.

FIG. 6 shows an example table 600 including example imaging and quantification parameters for one or more skin features. The imaging parameters may include, but not limited to, for each skin feature (column 602; e.g., pores), 1) region(s) of the body part that is used for measurement and/or quantification of the skin feature (column 606; e.g., two cheeks+nose+chin+(optional) forehead for pores); 2) type and number of poses used in the measurement and/or quantification of the skin feature (column, 608; e.g., frontal and two laterals of ¾ view for pores); and 4) light source (column 610, e.g., daylightRGB for pores). Further, quantification parameters may be used and example quantification parameters corresponding to each skin feature is shown in column 604. As a non-limiting example, the quantification parameters used in the evaluation of pores may include one or more of a pore number, a pore distribution, a pore occupancy ratio, a mean pore size, a variance of pore size, etc.

The quantification parameters may be used for evaluation of a certain conditions and/or feature, and thus, is based the condition and/or feature analyzed. As a non-limiting example, upon obtaining the electromagnetic spectrum profile, for evaluation of a wrinkle or a set of wrinkles, a length, a width, and a depth of the feature(s) identified as wrinkle may be evaluated from the electromagnetic spectrum profile (that shows response of the wrinkle to various light sources). While imaging data from a camera alone may provide length and width information, UV and/or IR imaging data may provide depth information, as well as pigmentation information of the wrinkles or area between the wrinkles, which provides more accurate skin condition and/or feature evaluation. Further, the changes in the wrinkles may be tracked over time, which may be used, for example, to determine if a current treatment is helpful in reducing one or more of a length, width, and/or depth of the wrinkle.

In one embodiment, provided herein is a smart mirror system comprising: a frame; a mirror coupled to the frame; a camera coupled to the frame; a sensor including an emitter and a detector; and one or more processors including executable instructions stored in one or more non-transitory memory devices that when executed cause the one or more processors to: acquire one or more imaging datasets via one or more of the camera and the sensor; and generate a skin profile based on the imaging datasets; and wherein, the detector includes light sensing capabilities in a visible region of the electromagnetic spectrum.

In a first example of the system, the detector further includes infrared light sensing capabilities.

In a second example of the system, which optionally includes the first example, the detector further includes ultraviolet light sensing capabilities.

In a third example of the system, which optionally includes one or more of the first and the second examples, the smart mirror system of claim 1, wherein the emitter comprises a UV light source and/or a visible light source.

In a fourth example of the system, which optionally includes one or more of the first through third examples, smart mirror system of claim 1, wherein the emitter comprises an infrared light source.

In a fifth example of the system, which optionally includes one or more of the first through fourth examples, the one or more processors include further instructions that when executed cause the one or more processors to: generate a plurality of pixel groups based on the one or more imaging datasets, wherein each pixel group comprises references to one or more sensor features.

In a sixth example of the system, which optionally includes one or more of the first through fifth examples, the one or more sensor features comprise visible and UV features, or visible and infrared features.

In a seventh example of the system, which optionally includes one or more of the first through sixth examples, the smart mirror system of claim 7, wherein the one or more processors include further instructions that when executed cause the one or more processors to: output a skin condition assessment of a skin condition based on the one or more sensor features.

In an eighth example of the system, which optionally includes one or more of the first through seventh examples, the smart mirror system of claim 8, wherein the one or more processors include further instructions that when executed cause the one or more processors to: display via augmented reality the assessment overlaid on a user's face image in the mirror.

In a ninth example of the system, which optionally includes one or more of the first through eighth examples, the UV light source emits ultraviolet light in a range between 315 and 400 nm wavelength.

In a tenth example of the system, which optionally includes one or more of the first through ninth examples, the UV light source emits ultraviolet light at 365 nm.

In an eleventh example of the system, which optionally includes one or more of the first through tenth examples, the skin condition includes one or more of a pore, spot, wrinkle, texture, redness, dark circle, eye bag, porphyrins, sebum, brown spot, and ultraviolet spot.

In another embodiment, provided herein is a smart mirror system comprising: a frame; a mirror coupled to the frame; a camera coupled to the frame; a first light source configured to output white light having wavelengths in a visible range; a second light source configured to output UV light having wavelengths in a UV range; and one or more processors including executable instructions stored in one or more non-transitory memory devices that when executed cause the one or more processors to: acquire one or more images of a user via the camera using a first lighting condition generated based on the first light source or the second light source; process the one or more images to detect one or more skin features; generate a skin profile analysis output based on the one or more skin features; and display, via a display portion of a user interface of the mirror, the skin profile analysis output.

In a first example of the system, the one or more skin features include one or more of pores, spots, wrinkles, texture, redness, dark circles, eye bags, sebum porphyrins, brown spots, and ultraviolet spots.

In a second example of the system, which optionally includes the first example, the skin profile analysis output comprises a quantification for each of the one or more skin features, the quantification including, for each of the one or more features, one or more of an average feature size, a percentage of skin area occupied by each feature, and a color distance distribution of each feature with respect to a baseline skin tone and color of the user.

In a third example of the system, which optionally includes one or more of the first and the second examples, the skin profile analysis output comprises an evolution of each of the one or more skin features over a duration of time; and wherein the evolution of each of the one or more skin features includes a change in one or more of an average feature size and a color shift over the duration of time.

In a fourth example of the system, which optionally includes one or more of the first through third examples, the evolution of each of the one or more skin features over the duration of time is evaluated based on one or more previous skin profile analysis of the user performed at one or more time points prior to acquisition of the one or more images of the user.

In a fifth example of the system, which optionally includes one or more of the first through fourth examples, process the one or more images to detect the one or more skin features comprises: identify, a body part for skin analysis in the one or more images; generate, a three-dimensional map of the body part using the one or more images; and mapping each of the one or more skin features on to the three-dimensional map of the body part.

In a sixth example of the system, which optionally includes one or more of the first through fifth examples, the body part includes one or more of a face or a portion thereof, a right hand or a portion thereof, a left hand or a portion thereof, an right arm or a portion thereof, a left arm or a portion thereof, a right leg or a portion thereof, a left leg or a portion thereof, and a trunk or a portion thereof.

In a seventh example of the system, which optionally includes one or more of the first through sixth examples, the one or more processors include further instructions that when executed cause the one or more processors to: display, via augmented reality, the one or more skin features overlaid on a user's image on the display portion of the mirror.

In an eighth example of the system, which optionally includes one or more of the first through seventh examples, the user's image is a three-dimensional image and/or a two dimensional image of the user generated from the one or more images.

In a ninth example of the system, which optionally includes one or more of the first through eighth examples, the one or more processors include further instructions that when executed cause the one or more processors to: classify, using a trained machine learning algorithm, one or more skin areas and one or more non-skin analysis areas; and detect, the one or more skin features based on the one or more skin areas.

In a tenth example of the system, which optionally includes one or more of the first through ninth examples, detect the one or more skin features based on the one or more skin areas comprises: encode an image comprising the one or more skin areas onto a four dimensional matrix based on a LAB color space; and detect the one or more skin features based on a color distance of the one or more skin features with respect to a base line skin tone in the LAB color space.

In an eleventh example of the system, which optionally includes one or more of the first through tenth examples, the system further comprises one or more thermopile sensors, the one or more thermopile sensors configured to acquire one or more of a skin temperature data and a body temperature data; and wherein the one or more processors include further instructions that when executed cause the one or more processors to: output a temperature profile of the user based on the skin temperature data; and output a body temperature of the user based on the body temperature.

In a twelfth example of the system, which optionally includes one or more of the first through eleventh examples, the one or more processors include further instructions that when executed cause the one or more processors to: determine, via one or more time of flight sensors, a distance of a user from the mirror; and responsive to the distance greater than a threshold, apply a distance compensation based on the distance to the one or more images for color correction.

In a thirteenth example of the system, which optionally includes one or more of the first through twelfth examples, each of the first light source and the second light source is a string of light emitting diodes (LEDs).

In a fourteenth example of the system, which optionally includes one or more of the first through thirteenth examples, the first light source is a set of correlated color temperature RGB LEDs; and wherein the second light source is a set of UV LEDs.

The smart mirror system of claim 1, wherein the first lighting condition is determined based on a type of the one or more skin features.

In another embodiment, provided herein is a smart mirror system comprising: a frame; a mirror coupled to the frame; a camera coupled to the frame; and a first light source configured to output white light having wavelengths in a visible range; a second light source configured to output UV light having wavelengths in a UV range; one or more processors including executable instructions stored in one or more non-transitory memory devices that when executed cause the one or more processors to: acquire a first set of images of a user via the camera using a first lighting condition provided by the first light source; acquire a second set of images of the user via the camera using a second lighting condition provided by the second light source; process the first set of images to obtain a first set of skin features; process the second set of images to obtain a second set of skin features; and generate a skin profile analysis output based on one or more of the first set of skin features and the second set of skin features.

In a first example of the system, the one or more processors include further instructions that when executed cause the one or more processors to: generate a plurality of pixel groups or voxel groups based on the first set of images and the second set of images, wherein each pixel or voxel group comprises references to at least one first skin feature in the first set of skin features and at least one second skin feature in the second set of skin features.

In a second example of the system, which optionally includes the first example, the smart mirror system of claim 16, wherein the one or more processors include further instructions that when executed cause the one or more processors to: generate a plurality of pixel groups or voxel groups based on the first set of images and the second set of images, wherein each pixel or voxel group includes skin feature information acquired using the first light source and/or the second light source.

In a fourth example of the system, which optionally includes one or more of the first through third examples, the one or more of first and second sets of skin features include one or more of pores, spots, wrinkles, texture, redness, dark circles, eye bags, sebum porphyrins, brown spots, and ultraviolet spots.

In a fifth example of the system, which optionally includes one or more of the first through fourth examples, the skin profile analysis output comprises a quantification for each of the one or more of the first and second sets of skin features, the quantification including, for each of the one or more features, one or more of an average feature size, a percentage of skin area occupied by each feature, and a color distance distribution of each feature with respect to a baseline skin tone and color of the user.

In a sixth example of the system, which optionally includes one or more of the first through fifth examples, the skin profile analysis output comprises an evolution of each of the one or more of the first and second sets of skin features over a duration of time; and wherein the evolution of each of one or more of the first and the second sets of skin features includes a change in one or more of an average feature size and a color shift over the duration of time.

In a seventh example of the system, which optionally includes one or more of the first through sixth examples, the evolution of each of the one or more of the first and the second set of skin features over the duration of time is evaluated based on one or more previous skin profile analysis of the user performed at one or more time points prior to acquisition of the first and second sets of images of the user.

In an eighth example of the system, which optionally includes one or more of the first through seventh examples, the one or more processors include further instructions that when executed cause the one or more processors to: display, via augmented reality, the one or more of the first and second sets of skin features overlaid on a user's image on the display portion of the mirror.

In a ninth example of the system, which optionally includes one or more of the first through eighth examples, each of the first light source and the second light source is a string of light emitting diodes (LEDs).

In a tenth example of the system, which optionally includes one or more of the first through ninth examples, the first light source is a set of correlated color temperature RGB LEDs; and wherein the second light source is a set of UV LEDs.

In another embodiment, provided herein is a method for performing skin analysis, the method comprises: acquiring, via a camera integrated with a smart mirror, one or more images of a user illuminated under a first lighting condition provided by a first light source coupled to the smart mirror; processing, via a processor of the smart mirror, the one or more images to classify one or more skin features; generating, via the processor, a skin profile analysis output based on the one or more skin features; and outputting, via a display portion of a user interface of the mirror, the skin profile analysis output.

In a first example of the method, the one or more skin features include one or more of pores, spots, wrinkles, texture, redness, dark circles, eye bags, sebum porphyrins, brown spots, and ultraviolet spots.

In a second example of the method, which optionally includes the first example, the skin profile analysis output comprises a quantification for each of the one or more skin features, the quantification including, for each of the one or more features, one or more of an average feature size, a percentage of skin area occupied by each feature, and a color distance distribution of each feature with respect to a baseline skin tone and color of the user.

In a third example of the method, which optionally includes one or more of the first and the second examples, the skin profile analysis output comprises an evolution of each of the one or more skin features over a duration of time; and wherein the evolution of each of the one or more skin features includes a change in one or more of an average feature size and a color shift over the duration of time.

In a fourth example of the method, which optionally includes one or more of the first through third examples, the evolution of each of the one or more skin features over the duration of time is evaluated based on one or more previous skin profile analysis of the user performed at one or more time points prior to acquisition of the one or more images of the user.

In a fifth example of the method, which optionally includes one or more of the first through fourth examples, processing the one or more images to detect the one or more skin features comprises identifying, a body part for skin analysis in the one or more images; and generating a three-dimensional map of the body part using the one or more images; and mapping each of the one or more skin features on to a pixel or voxel group in the three-dimensional map of the body part.

In this way, the methods and smart mirror systems described herein allow generation of a skin profile of a user which includes skin features detected under different lighting conditions. As a result, a comprehensive skin profile is generated. Further, the skin features are correlated time to track evolution of the skin features over time. Furthermore, the skin analysis is performed such that a given skin feature is compared with respect to surrounding baseline skin tones, color, and hues. As a result, the systems and methods described herein can be applied for evaluate skin profile of a variety of skin colors, tones, and hues, with improved accuracy and efficiency. Thus, the systems and methods for smart mirror provide a significant improvement in the area of smart mirror and skin analysis using smart mirror.

Referring now to FIG. 9 it shows an example illustration of a smart mirror system that may be used for skin analysis. The smart mirror system 10 includes features the skin analysis system 100 described at FIG. 1. The system 10 includes a mirror 12. The mirror 12 can be mounted on a base 26. The mirror 12 could also be directly mounted in a counter, a wall, or any other structure. The electronic display 14 is mounted on, coupled to, or otherwise disposed on a first side of the mirror 12, while a sensor frame 28 containing the one or more sensors 16 is disposed at an opposing second side of the mirror 12. The side of the mirror 12 where the display 14 is located is generally referred to as the display-side of the mirror 12. The side of the mirror 12 where the sensor frame 28 is located is generally referred to as the user-side of the mirror 12, as this is the side of the mirror 12 where the user will be located during operation.

The electronic display 14 is generally mounted in close proximity to the surface of the display-side of the mirror 12. The electronic display 14 can be any suitable device, such as an LCD screen, an LED screen, a plasma display, an OLED display, a CRT display, an LED dot matrix display, or the like. In some implementations, the LED dot matrix display is a relatively low resolution LED display. For example, the relatively low resolution LED display can include between about 1 and about 5 LEDs per square inch, between about 1 and about 10 LEDs per square inch, between about 1 and about 25 LEDs per square inch, or between about 1 and about 50 LEDs per square inch. Due to the partially reflective nature of the mirror 12, when the display 14 is activated (e.g. turned on and emitting light to display an image), a user standing on the user-side of the mirror 12 is able to view any portion of the display 14 that is emitting light through the mirror 12. When the display 14 is turned off, light that is incident on the user-side of the mirror 12 from the surroundings will be partially reflected and partially transmitted. Because the display 14 is off, there is no light being transmitted through the mirror 12 to the user-side of the mirror 12 from the display-side. Thus, the user standing in front of the mirror 12 will see their reflection due to light that is incident on the user-side of the mirror 12 and is reflected off of the mirror 12 back at the user. When the display 14 is activated, a portion of the light produced by the display 14 that is incident on the mirror 12 from the display-side is transmitted through the mirror 12 to the user-side. The mirror 12 and the display 14 are generally configured such that the intensity of the light that is transmitted through the mirror 12 from the display 14 at any given point is greater than the intensity of any light that is reflected off of that point of the mirror 12 from the user-side. Thus, a user viewing the mirror 12 will be able to view the portions of the display 14 that are emitting light, but will not see their reflection in the portions of those mirror 12 through which the display light is being transmitted.

The electronic display 14 can also be used to illuminate the user or other objects that are located on the user-side of the mirror 12. The processor 22 can activate a segment of the display 14 that generally aligns with the location of the object relative to the mirror 12. In an implementation, this segment of the display 14 is activated responsive to one of the one or more sensors 16 detecting the object and its location on the user-side of the mirror 12. The segment of the display 14 can have a ring-shaped configuration which includes an activated segment of the display 14 surrounding a non-activated segment of the display 14. The non-activated segment of the display 14 could be configured such that no light is emitted, or could be configured such that some light is emitted by the display 14 in the non-activated segment, but it is too weak or too low in intensity to be seen by the user through the mirror 12. In an implementation, the activated segment of the display 14 generally aligns with an outer periphery of the object, while the non-activated segment of the display 14 generally aligns with the object itself. Thus, when the object is a user's face, the user will be able to view the activated segment of the display 14 as a ring of light surrounding their face. The non-activated segment of the display 14 will align with the user's face, such that the user will be able to see the reflection of their face within the ring of light transmitted through the mirror 12. In another implementation, the non-activated segment of the display 14 aligns with the object, and the entire remainder of the display 14 is the activated segment. In this implementation, the entire display 14 is activated except for the segment of the display 14 that aligns with the object.

Generally, the system 10 includes one or more sensors 16 disposed in the sensor frame 28. The sensor frame 28 is mounted on, couple to, or otherwise disposed at the second side (user-side) of the mirror 12. The sensors 16 are generally located within a range of less than about five inches from the user-side surface of the mirror 12. In other implementations, the sensors 16 could be disposed between further away from the surface of the mirror 12, such as about between about 5 inches and about 10 inches. The sensors 16 are configured to detect the presence of a hand, finger, face, or other body part of the user when the user is within a threshold distance from the mirror 12. This threshold distance is the distance that the sensors 16 are located away from the user-side surface of the mirror 12. The sensors 16 are communicatively coupled to the processor 22 and/or memory 24. When the sensors 16 detect the presence of the user aligned with a certain point of the mirror 12 (and thus the display 14), the processor 22 is configured to cause the display 14 to react as if the user had touched or clicked the display 14 at a location on the display 14 corresponding to the point of the mirror 12. Thus, the sensors 16 are able to transform the mirror 12/display 14 combination into a touch-sensitive display, where the user can interact with and manipulate applications executing on the display 14 by touching the mirror 12, or even bringing their fingers, hands, face, or other body part in close proximity to the user-side surface of the mirror 12. In some implementations, the sensors 16 can include a microphone that records the user's voice. The data from the microphone can be sent to the processor 22 to allow the user to interact with the system 10 using their voice.

The one or more sensors 16 are generally infrared sensors, although sensors utilizing electromagnetic radiation in other portions of the electromagnetic spectrum could also be utilized. In some examples, additionally or alternatively, one or more thermopile sensors (not shown) may be coupled to the system 10 in order to detect a temperature of a user. In some examples, the thermopile sensor may be coupled to the sensor frame.

The sensor frame 28 can have a rectangular shape, an oval shape, a circular shape, a square shape, a triangle shape, or any other suitable shape. In an implementation, the shape of the sensor frame 28 is selected to match the shape of the mirror 12. For example, both the mirror 12 and the sensor frame 28 can have rectangular shapes. In another implementation, the sensor frame 28 and the mirror 12 have different shapes. In an implementation, the sensor frame 28 is approximately the same size as the mirror 12 and generally is aligned with a periphery of the mirror 12. In another implementation, the sensor frame 28 is smaller than the mirror 12, and is generally aligned with an area of the mirror 12 located interior to the periphery of the mirror 12. In a further implementation, the sensor frame 28 could be larger than the mirror 12.

In an implementation, the mirror 12 generally has a first axis and a second axis. The one or more sensors 16 are configured to detect a first axial position of an object interacting with the sensors 16 relative to the first axis of the mirror 12, and a second axial position of the object interacting with the sensors 16 relative to the second axis of the mirror 12. In an implementation, the first axis is a vertical axis and the second axis is a horizontal axis. Thus, in viewing the sensor frame 28 from the perspective of the user, the sensor frame 28 may have a first vertical portion 28A and an opposing second vertical portion 28B, and a first horizontal portion 28C and an opposing second horizontal portion 28D. The first vertical portion 28A has one or more infrared transmitters disposed therein, and the second vertical portion 28B has one or more corresponding infrared receivers disposed therein. Each individual transmitter emits a beam of infrared light that is received by its corresponding individual receiver. When the user places a finger in close proximity to the mirror 12, the user's finger can interrupt this beam of infrared light such that the receiver does not detect the beam of infrared light. This tells the processor 22 that the user has placed a finger somewhere in between that transmitter/receiver pair. In an implementation, a plurality of transmitters is disposed intermittently along the length of the first vertical portion 28A, while a corresponding plurality of receivers is disposed intermittently along the length of the second vertical portion 28B. Depending on which transmitter/receiver pairs detect the presence of the user's finger (or other body part), the processor 22 can determine the vertical position of the user's finger relative to the display 14. The first axis and second axis of the mirror 12 could be for a rectangular-shaped mirror, a square-shaped mirror, an oval-shaped mirror, a circle-shaped mirror, a triangular-shaped mirror, or any other shape of mirror.

The sensor frame 28 similarly has one or more infrared transmitters disposed intermittently along the length of the first horizontal portion 28C, and a corresponding number of infrared receivers disposed intermittently along the length of the second horizontal portion 28D. These transmitter/receiver pairs act in a similar fashion as to the ones disposed along the vertical portions 28A, 28B of the sensor frame 28, and are used to detect the presence of the user's finger and the horizontal location of the user's finger relative to the display 14. The one or more sensors 16 thus form a two-dimensional grid parallel with the user-side surface of the mirror 12 with which the user can interact, and where the system 10 can detect such interaction.

In other implementations, the sensor frame 28 may include one or more proximity sensors, which can be, for example, time of flight sensors. Time of flight sensors do not rely on separate transmitters and receivers, but instead measure how long it takes an emitted signal to reflect off on an object back to its source. A plurality of proximity sensors on one edge of the sensor frame 28 can thus be used to determine both the vertical and horizontal positions of an object, such as the user's hand, finger, face, etc. For example, a column of proximity sensors on either the left or right edge can determine the vertical position of the object by determining which proximity sensor was activated, and can determine the horizontal position by using that proximity sensor to measure how far away the object is from the proximity sensor. Similarly, a row of proximity sensors on either the top or bottom edge can determine the horizontal position of the object by determining which proximity sensor was activated, and can determine the vertical position by using that proximity sensor to measure how far away the object is from the proximity sensor. When one or more of the proximity sensors detect the presence of the user's finger or other body part (e.g., hand, face, arm, etc.), the user's finger or other body part is said to be in the “line of sight” of the sensor.

Further, in some implementations, additionally or alternatively, one or more sensors may be used to determine a distance of the user with respect to the system 10. For example, the distance of the user may be determined based on a time difference between an emission of a signal and its return after being reflected from the user. In one example, the one or more sensors may be configured to emit IR light and detect reflected light from the user. Based on the distance of the user with respect to the system 10, a distance compensation model may be applied, via processor 22, to adjust one or more parameters of the system responsive to the distance. In one example, responsive to the distance of the user being greater than a first threshold distance, an intensity of light for illuminating the user may be increased. In another example, responsive to the distance of the user being greater than a second threshold distance, the second threshold distance greater than the first threshold distance, an indication may be provided to the user suggesting to move to be closer to the system. Further still, responsive to the distance of the user being greater than the first threshold distance but less than the second threshold distance, a distance compensation model may be applied to adjust for one or more deviations caused due to the distance being greater than the first threshold distance.

Further, in some implementations, one or more thermopile sensors may be used to detect a temperature of various skin areas of the user. In some examples, in addition to skin analysis performed used one or more images acquired via the camera coupled to the system, as discussed above, a temperature profile of the skin may be analyzed using one or more thermopile sensors to perform skin analysis. In some examples, the one or more thermopile sensors may be used to determine an overall user body temperature, and output an indication of the body temperature, via a display portion of the mirror 12.

The sensors 16 in the sensor frame 28 (whether IR transmitter/receiver pairs and/or proximity sensors) can be used by the system 10 to determine different types of interactions between the user and the system 10. For example, the system 10 can determine whether the using is swiping horizontally (left/right), vertically (up/down), diagonally (a combination of left/right and up/down), or any combination thereof. The system 10 can also detect when the user simply taps somewhere instead of swiping. In some implementations, the sensor frame 28 is configured to detect interactions between the user and the system 10 when the user is between about 3 centimeters and about 15 centimeters from the surface of the mirror 12. A variety of different applications and programs can be run by the processor 22, including touch-based applications designed for use with touch screens, such as mobile phone applications. Generally, any instructions or code in the mobile phone application that rely on or detect physical contact between a user's finger (or other body part) and the touch-sensitive display of the mobile phone (or other device) can be translated into instructions or code that rely on or detect the user's gestures in front of the mirror 12.

Any applications that are run by the processor 22 and are displayed by the display 14 can be manipulated by the user using the sensors 16 in the sensor frame 28. Generally, the sensors 16 in the sensor frame 28 detect actions by the user, which causes the processor 22 to take some action. For example, a user-selectable icon may be displayed on the display 14. The user can select the user-selectable icon, which triggers the processor 22 to take some corresponding action, such as displaying a new image or screen on the display 14. The triggering of the processor 22 due to the user's interaction with the system 10 can be affected using at least two different implementations.

In the first implementation, the processor 22 is triggered to take some action once the user's finger (or other body part) is removed from the proximity of the display 14 and/or the sensors 16, e.g., is removed from the line of sight of the sensors 16. For example, when the user wishes to select a user-selectable icon displayed on the display 14, they can move their finger into close proximity with the displayed user-selectable icon without touching the mirror 12, such that the user's finger is in the line of sight of the sensors 16. However, coming into the line of sight of the sensors 16 does not trigger the processor 22 to take any action, e.g., the user has not yet selected the icon by placing their finger near the icon on the display 14. Once the user removes their finger from the proximity of the icon on the display 14 and the line of sight of the sensor 16, the processor 22 is triggered to take some action. Thus, the user only selects the user-selectable icon once the user removes their finger from the proximity of the icon.

In the second implementation, the processor 22 is triggered to take some action when the user's finger (or other body part) is moved into proximity of the display 14 and/or the sensors 16, e.g., is moved into the line of sight of the sensors 16. In this implementation, as soon as the user moves their finger into close proximity of a user-selectable icon on the display 14 (e.g., moves their finger into the line of sight of the sensors 16), the processor 22 is triggered to take some action corresponding to the section of the icon. Thus, the user does not have to move their finger near the icon/sensors 16 and then remove their finger in order to select the icon, but instead only needs to move their finger near the icon/sensors 16. In some such implementations, selection of an icon requires that the user hold its finger near the icon for a predetermined amount of time (e.g., 1 second, 1.5 seconds, 2 seconds, 3 seconds, etc.).

Other methods of controlling the system 10 can also be used. For example, the system 10 can include a microphone (or communicate with a device that includes a microphone) to allow for voice control of the system 10. The system 10 can also include one or more physical buttons or controllers the user can physically actuate in order to interact with and control the system 10. In another example, the system 10 could communicate with a separate device that the user interacts with (such as a mobile telephone, tablet computer, laptop computer, desktop computer, etc.) to control the system 10. In some such implementations, the user's smart phone and/or tablet can be used as an input device to control the system 10 by mirroring the display 14 of the system 10 on a display of the smart phone and/or tablet and allowing the user to control the system 10 by touching and/or tapping the smart phone and/or tablet directly. The system 10 can also include one or more speakers to play music, podcasts, radio, or other audio. The one or more speakers can also provide the user feedback or confirmation of certain actions or decisions.

The system 10 further includes one or more light sources 18. In an implementation, the light sources 18 are light emitting diodes (LEDs) having variable color and intensity values that can be controlled by the processor 22. In other implementations, the light sources 18 can be incandescent light bulbs, halogen light bulbs, fluorescent light bulbs, black lights, UV light sources, discharge lamps, or any other suitable light source. The light sources 18 can be coupled to or disposed within the base 26 of the system 10, or they can be coupled to or disposed within the sensor frame 28. For example, while FIG. 9 only shows two light sources 18 disposed in a bottom portion of the system 10, a plurality of light sources 18 could be disposed about the frame such that the light sources 18 generally surround the mirror 12. In some implementations, the light sources 18 may be disposed on either the user-side of the mirror 12 or the display-side of the mirror 12. When disposed on the user-side of the mirror 12, the light emitted by the light sources 18 is configured to travel through the mirror 12 towards the user. The light sources 18 can also be rotationally or translationally coupled to the sensor frame 28 or other parts of the system 10 such that the light sources 18 can be physically adjusted by the user and emit light in different directions. The light sources 18 could also be disposed in individual housings that are separate from both the mirror 12 and the display 14. The light sources 18 are configured to produce light that is generally directed outward away from the mirror 12 and toward the user. The light produced by the one or more light sources 18 can thus be used to illuminate the user (or any other object disposed on the user-side of the mirror 12). Because they are variable in color and intensity, the light sources 18 can thus be used to adjust the ambient light conditions surrounding the user.

The system 10 also includes one or more cameras 20 mounted on or coupled to the mirror 12. The cameras 20 could be optical cameras operating using visible light, infrared (IR) cameras, RGB cameras, three-dimensional (depth) cameras, or any other suitable type of camera. The one or more cameras 20 are disposed on the display-side of the mirror 12. In an implementation, the one or more cameras 20 are located above the electronic display 14, but are still behind the mirror 12 from the perspective of the user. The lenses of the one or more cameras 20 faces toward the mirror 12 and are thus configured to monitor the user-side of the mirror 12. In an implementation, the one or more cameras 20 monitor the user-side of the mirror 12 through the partially reflective coating on the mirror 12. In another implementation, the one or more cameras 20 are disposed at locations of the mirror 12 where no partially reflective coating exists, and thus the one or more cameras 20 monitor the user-side of the mirror 12 through the remaining transparent material of the mirror 12. The one or more cameras 20 may be stationary, or they may be configured to tilt side-to-side and up and down. The cameras 20 can also be moveably mounted on a track and be configured to move side-to-side and up and down. The one or more cameras 20 are configured to capture still images or video images of the user-side of the mirror 12. The display 14 can display real-time or stored still images or video images captured by the one or more cameras 20.

The one or more cameras 20 are communicatively coupled to the processor 22. The processor 22 can run an object recognition (OR) algorithm that utilizes principles of computer vision to detect and identify a variety of objects based on the still or video images captured by the one or more cameras 20. The processor 22 can be configured to modify the execution of an application being executing by the processor 22, such as automatically launching a new application or taking a certain action in an existing application, based on the object that is detected and identified by the cameras 20 and the processor 22. For example, following the detection of an object in the user's hand and the identification of that object as a toothbrush, the processor 22 can be configured to automatically launch a tooth-brushing application to run on the display 14, or launch a tooth brushing feature in the current application.

In some implementations, the processor 22 may be configured to automatically perform skin analysis responsive to detecting the presence of a user within a threshold distance from the mirror, as discussed above with respect to FIGS. 2-8E, and output and/or one or more skin analysis results. In some examples, responsive to a request for skin analysis from the user, the processor 22 may begin image acquisition, via the camera 20, and perform skin analysis on the acquired images.

The processor 22 can be configured to automatically launch an application to assist the user in shaving upon detecting and identifying a razor, or an application to assist the user in applying makeup upon detecting and identifying any sort of makeup implement, such as lipstick, eye shadow, etc. The one or more cameras 20 can also recognize faces of users and differentiate between multiple users. For example, the camera 20 may recognize the person standing in front of the mirror 12 and execute an application that is specific to that user. For example, the application could display stored data for that user, or show real-time data that is relevant to the user.

In an implementation, the processor 22 can be configured to execute a first application while the display 14 displays a first type of information related to the first application. Responsive to the identification of the object by the system 10, the processor is configured to cause the display 14 to display a second type of information related to the first application, the second type of information being (i) different from the first type of information and (ii) based on the identified object. In another implementation, responsive to the identification of the object, the processor is configured to execute a second application different from the first application, the second application being based on the identified object.

In some implementations, the components of the system 10 are generally waterproof, which allows the system 10 to more safely operate areas where water or moisture may be present, such as bathrooms. For example, a waterproof sealing mechanism may be used between the mirror 12 and the sensor frame 28 to ensure that moisture cannot get behind the mirror 12 to the electronic components. This waterproof sealing mechanism can include a waterproof adhesive, such as UV glue, a rubber seal surrounding the periphery of the mirror 12 on both the user-side of the mirror 12 and the display-side of the mirror, or any other suitable waterproof sealing mechanism. Further, a waterproof or water-repellant covering can be placed over or near any components of the system 10 that need to be protected, such as speakers or microphones. In some implementations, this covering is a water-repellant fabric.

The components of the system 10 can also be designed to increase heat dissipation. Increased heat dissipation can be accomplished in a number of ways, including by using a reduced power supply or a specific power supply form factor. The system 10 can also include various mechanisms for distributing heat from behind the mirror 12, including heat sinks and fans. In some implementations, the electronic components (such as display 14, sensors 16, light sources 18, camera 20, processors 22, and memory 24) are all modular so that the system 10 can easily be customized.

In addition to the sensors 16 described herein, the smart mirror system 10 may include one or more UV imaging sensors, one or more IR imaging sensors, and/or one or more RGB imaging sensors, as described above with respect to FIG. 1A.

FIG. 10A illustrates a front elevation view of the system 10, while FIG. 10B illustrates a side elevation view of the system 10. As can be seen in FIG. 10A, the sensor frame 28 surrounds the mirror 12, while portions of the display 14 that are activated are visible through the mirror 12. FIG. 10A also shows the two-dimensional grid that can be formed by the sensors 16 in the sensor frame 28 that is used to detect the user's finger, head, or other body part. This two-dimensional grid is generally not visible to the user during operation. FIG. 10B shows the arrangement of the sensor frame 28 with the sensors 16, the mirror 12, the display 14, and the camera 20. In an implementation, the processor 22 and the memory 24 can be mounted behind the display 14. In other implementations, the processor 22 and the memory 24 may be located at other portions within the system 10, or can be located external to the system 10 entirely. The system 10 generally also includes housing components 43A, 43B that form a housing that contains and protects the display 14, the camera 20, and the processor 22.

It is contemplated that any elements of any one of the above alternative implementations can be combined with any elements of one or more of any of the other alternative implementations and such combinations are intended as being included in the scope of the present disclosure.

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Certain embodiments of this application are described herein. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

Particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A smart mirror system comprising:
a frame;
a mirror coupled to the frame;
a camera coupled to the frame;
a first light source configured to output white light having wavelengths in a visible range;
a second light source configured to output UV light having wavelengths in a UV range; and
one or more processors including executable instructions stored in one or more non-transitory memory devices that when executed cause the one or more processors to:
acquire one or more images of a user via the camera using a first lighting condition generated based on the first light source or the second light source;
process the one or more images to detect one or more skin features;
generate a skin profile analysis output based on the one or more skin features; and
display, via a display portion of a user interface of the mirror, the skin profile analysis output.

2. The smart mirror system of claim 1, wherein the one or more skin features include one or more of pores, spots, wrinkles, texture, redness, dark circles, eye bags, sebum porphyrins, brown spots, and ultraviolet spots.

3. The smart mirror system of claim 1, wherein the skin profile analysis output comprises a quantification for each of the one or more skin features, the quantification including, for each of the one or more features, one or more of an average feature size, a percentage of skin area occupied by each feature, and a color distance distribution of each feature with respect to a baseline skin tone and color of the user.

4. The smart mirror system of claim 1, wherein the skin profile analysis output comprises an evolution of each of the one or more skin features over a duration of time; and wherein the evolution of each of the one or more skin features includes a change in one or more of an average feature size and a color shift over the duration of time.

5. The smart mirror system of claim 1, wherein the evolution of each of the one or more skin features over the duration of time is evaluated based on one or more previous skin profile analysis of the user performed at one or more time points prior to acquisition of the one or more images of the user.

6. The smart mirror system of claim 1, wherein process the one or more images to detect the one or more skin features comprises:
identify, a body part for skin analysis in the one or more images;
generate, a three-dimensional map of the body part using the one or more images; and mapping each of the one or more skin features on to the three-dimensional map of the body part.

7. The smart mirror system of claim 1, wherein the body part includes one or more of a face or a portion thereof, a right hand or a portion thereof, a left hand or a portion thereof, a right arm or a portion thereof, a left arm or a portion thereof, a right leg or a portion thereof, a left leg or a portion thereof, and a trunk or a portion thereof.

8. The smart mirror system of claim 1, wherein the one or more processors include further instructions that when executed cause the one or more processors to:
display, via augmented reality, the one or more skin features overlaid on a user's image on the display portion of the mirror.

9. The smart mirror system of claim 1, wherein the one or more processors include further instructions that when executed cause the one or more processors to:
classify, using a trained machine learning algorithm, one or more skin areas and one or more non-skin analysis areas; and
detect, the one or more skin features based on the one or more skin areas.

10. The smart mirror system of claim 9, wherein detect the one or more skin features based on the one or more skin areas comprises:
encode an image comprising the one or more skin areas onto a four dimensional matrix based on a LAB color space; and
detect the one or more skin features based on a color distance of the one or more skin features with respect to a base line skin tone in the LAB color space.

11. The smart mirror system of claim 1, further comprising one or more thermopile sensors, the one or more thermopile sensors configured to acquire one or more of a skin temperature data and a body temperature data; and wherein the one or more processors include further instructions that when executed cause the one or more processors to: output a temperature profile of the user based on the skin temperature data; and output a body temperature of the user based on the body temperature.

12. The smart mirror system of claim 1, wherein the one or more processors include further instructions that when executed cause the one or more processors to:
determine, via one or more time of flight sensors, a distance of a user from the mirror; and responsive to the distance greater than a threshold, apply a distance compensation based on the distance to the one or more images for color correction.

13. The smart mirror system of claim 1, wherein each of the first light source and the second light source is a string of light emitting diodes (LEDs).

14. The smart mirror system of claim 1, wherein the first light source is a set of correlated color temperature RGB LEDs; and wherein the second light source is a set of UV LEDs.

15. The smart mirror system of claim 1, wherein the first lighting condition is determined based on a type of the one or more skin features.

16. A smart mirror system comprising:
a frame;
a mirror coupled to the frame;
a camera coupled to the frame; and
a first light source configured to output white light having wavelengths in a visible range;
a second light source configured to output UV light having wavelengths in a UV range; and
one or more processors including executable instructions stored in one or more non-transitory memory devices that when executed cause the one or more processors to:
acquire a first set of images of a user via the camera using a first lighting condition provided by the first light source;
acquire a second set of images of the user via the camera using a second lighting condition provided by the second light source;
process the first set of images to obtain a first set of skin features;
process the second set of images to obtain a second set of skin features; and
generate a skin profile analysis output based on one or more of the first set of skin features and the second set of skin features.

17. The smart mirror system of claim 16, wherein the one or more processors include further instructions that when executed cause the one or more processors to:
generate a plurality of pixel groups or voxel groups based on the first set of images and the second set of images, wherein each pixel or voxel group comprises references to at least one first skin feature in the first set of skin features and at least one second skin feature in the second set of skin features.

18. The smart mirror system of claim 16, wherein the one or more processors include further instructions that when executed cause the one or more processors to:
generate a plurality of pixel groups or voxel groups based on the first set of images and the second set of images, wherein each pixel or voxel group includes skin feature information acquired using the first light source and/or the second light source.

19-25. (canceled)

26. A method for performing skin analysis, the method comprises:
acquiring, via a camera integrated with a smart mirror, one or more images of a user illuminated under a first lighting condition provided by a first light source coupled to the smart mirror;
processing, via a processor of the smart mirror, the one or more images to classify one or more skin features;
generating, via the processor, a skin profile analysis output based on the one or more skin features; and
outputting, via a display portion of a user interface of the mirror, the skin profile analysis output.

27-30. (canceled)

31. The method of claim 26, wherein processing the one or more images to detect the one or more skin features comprises identifying, a body part for skin analysis in the one or more images; and generating a three-dimensional map of the body part using the one or more images; and mapping each of the one or more skin features on to a pixel or voxel group in the three-dimensional map of the body part.
